(19) United States
(12) Patent Application Publication
(10) Pub. No.: US 2021/0088505 A1
(43) Pub. Date: Mar. 25, 2021

(54) Unipotent Neutrophil Progenitor Cells, Methods of Preparation, and Uses Thereof

(71) Applicant: La Jolla Institute for Allergy and Immunology, La Jolla, CA (US)

(72) Inventors: Yanfang Peipei ZHU, San Diego, CA (US); Catherine Lynn HEDRICK, San Diego, CA (US)

(21) Appl. No.: 16/603,522

(22) PCT Filed: Apr. 6, 2018
(86) PCT No.: PCT/US2018/026613

§ 371 Date: Oct. 7, 2019

Related U.S. Application Data
(60) Provisional application No. 62/483,305, filed on Apr. 7, 2017.

(51) Int. Cl. G01N 33/50; G01N 33/574; G01N 33/569; A61K 35/15

(52) U.S. Cl. CPC G01N 33/5047; G01N 33/574; G01N 2800/52; A61K 35/15; G01N 33/56972

(57) ABSTRACT

The present invention relates to a unipotent neutrophil progenitor population, to methods of preparing and using same. In certain embodiments, the neutrophil progenitor population have at least the phenotype CD45+, CD41−, CD127(IL-7Rα)−, CD19−, CD3−, CD161 (NK1.1)−, CD169 (Siglec 1)−, CD11c−, Siglec 8−, FcεRIα− and CD115 (CSF-1R)−.

### CROSS REFERENCE TO RELATED APPLICATIONS

The present application claims the benefit of U.S. provisional patent application Ser. No. 62/483,305 filed on Apr. 7, 2017 by Catherine Hedrick. The contents of the above-referenced document are incorporated herein by reference in their entirety.

### STATEMENT REGARDING FEDERALLY SPONSORED RESEARCH OR DEVELOPMENT

This invention was made with government support under grant numbers R01HL134236, P01HL136275, R01CA202987, 1S100D018499-01 and ADA7-12-MN-31(04) awarded by the National Institute of Health. The U.S. Government has certain rights in the invention.

### TECHNICAL FIELD

This application generally relates to the field of progenitor cells and, more specifically, to neutrophil progenitor cells, methods of preparation and use thereof.

### BACKGROUND

Neutrophils represent the most abundant cell population in the innate immune system and are indispensable antagonists of microbial infection and facilitators of wound healing. More recently, the role of neutrophils has also been extended to cover immune-related conditions such as cancer (1-3). Indeed, a number of studies have suggested that neutrophils may have both pro- and anti-tumorigenic roles, which apparently differs with cancer type and disease stage (Treffers et al., Immunol. Rev. 2016 September; 273(1):312-28). Additionally, studies have also suggested that tumors may manipulate neutrophils, sometimes early in their differentiation process, to create diverse phenotypic and functional polarization states able to alter tumor behavior (Coffelt et al., Nature Reviews Cancer 16, 431-446, 2016).

Studies have reported that substantially increased numbers of neutrophils are found in the blood of many patients with advanced cancer, and that this is often associated with poor prognosis as has been demonstrated in various types of cancer, including melanoma, renal cancer, and lung cancer. The neutrophil-to-lymphocyte ratio (NLR) was introduced more recently to represent probably, at least in most cases, the same phenomenon, and this appears to be an even better predictor for poor disease and treatment outcome.

Although a high NLR appears associated with an increase in markers of a systemic inflammatory response, including elevated circulating concentrations of G-CSF, IL-8, MIP1, and PDGF, the biological mechanisms leading to an elevated NLR in cancer patients are still largely unknown.

Neutrophils and monocytes arise from the same progenitor cells, the Granulocyte Monocyte Progenitor (GMP) in the bone marrow (BM). In mouse BM, it is known that Hematopoietic Stem and Progenitor Cells (HSPCs) commit to a series of checkpoints for lineage decision from the Long-Term and Short-Term Hematopoietic Stem Cells (LT/ST-HSCs) into the Common Myeloid Progenitor (CMP) for myeloid cell production. CMPs give rise to both megakaryocyte-erythrocyte progenitors (MEPs) and GMPs (5). GMPs are the oligopotent progenitors for granulocytes, monocytes, macrophages, and dendritic cells (DCs) (6) and are reprogrammed in cancer to produce tumor-associated monocytes and neutrophils (7, 8). Unipotent neutrophil progenitor cells, however, have not yet been identified, therefore, specific studies of neutrophil biology in health and disease have been impeded.

In light of at least the above, there is a need to better understand the biological mechanisms underlying neutrophil differentiation biology in health and disease.

### SUMMARY

This Summary is provided to introduce a selection of concepts in a simplified form that are further described below in the Detailed Description. This Summary is not intended to identify key aspects or essential aspects of the claimed subject matter.

The present disclosure aims to at least identify, characterize and/or isolate unipotent neutrophil progenitor cells.

The present disclosure relates broadly to a method of treating a subject, wherein the method comprises i) processing a biological sample from the subject, the sample being suspected of including neutrophil cells to determine a concentration level thereof, ii) comparing the concentration level to a reference level, and iii) treating said subject at least based on said comparison, the treating step including stimulating or inhibiting differentiation of unipotent neutrophil progenitor cells into neutrophil cells so as to modulate the concentration of said neutrophil cells in said subject.

In certain aspects of the present invention, a method for evaluating a condition status in a subject is provided, the condition being associated with neutropenia. The method comprises providing a biological sample from said subject, the sample being suspected of including unipotent neutrophil progenitor cells in said sample. The method further includes processing the sample to determine a concentration or activation level of said unipotent neutrophil progenitor cells in said sample. Optionally, the method may further include comparing the concentration or activation level to a reference level, and evaluating the condition status based on at least the comparison, the condition being associated with neutropenia.

In certain alternative aspects of the present invention, a method for evaluating cancer in a subject is provided, the method comprising providing a biological sample from said subject, the sample being suspected of including unipotent neutrophil progenitor cells. The method further includes processing the sample to determine a concentration or activation level of said unipotent neutrophil progenitor cells in said sample. Optionally, the method may further include comparing the concentration or activation level to a reference level, and evaluating the subject as having or not having cancer based on at least the comparison.

In certain aspects of the present invention, a method for determining response or resistance to cancer treatment in a subject undergoing cancer treatment is provided. The method comprises providing a biological sample from said subject, the sample being suspected of including unipotent neutrophil progenitor cells. The method further includes processing the sample to determine a concentration or activation level of said unipotent neutrophil progenitor cells in said sample. Optionally, the method may further include comparing the concentration or activation level to a reference level, and evaluating the response or resistance to the cancer treatment based on at least the comparison.

In other aspects of the present invention, a method for determining response to a treatment for a condition associated with neutropenia in a subject undergoing the treatment is provided. The method comprises providing a biological sample from said subject, the sample being suspected of including unipotent neutrophil progenitor cells. The method further includes processing the sample to determine a concentration or activation level of said unipotent neutrophil progenitor cells in said sample. Optionally, the method may further include comparing the concentration or activation level to a reference level, and evaluating the response or resistance to the treatment based on at least the comparison.

In alternative aspects of the present invention, a method of reducing risk of cancer progression or cancer relapse in a subject is provided, the method comprising i) providing a biological sample form said subject, the sample being suspected of including unipotent neutrophil progenitor cells, ii) processing the sample to determine a concentration or activation level of said unipotent neutrophil progenitor cells in said sample, iii) comparing the concentration or activation level to a reference level, and iv) selectively administering a cancer therapeutic agent at least based on the comparison in step (iii) so as to reduce risk of cancer progression or cancer relapse in the subject.

In certain aspects of the present invention, a method of reducing risk of a condition associated with neutropenia in a subject is provided, the method comprising i) providing a biological sample from said subject, the sample being suspected of including unipotent neutrophil progenitor cells, ii) processing the sample to determine a concentration or activation level of said unipotent neutrophil progenitor cells in said sample, iii) comparing the concentration or activation level to a reference level, and iv) selectively administering a therapeutic agent at least based on the comparison in step (iii) so as to reduce risk of the condition associated with neutropenia in the subject.

In certain other aspects of the present invention, a method for screening a candidate molecule for an activity on cell differentiation of unipotent neutrophil progenitor cells into neutrophils is provided. The method comprises i) contacting said unipotent neutrophil progenitor cells with the candidate molecule, and ii) determining the activity of the candidate molecule on the cell differentiation of said unipotent cells into neutrophils.

In alternative aspects of the present invention, a method for screening a candidate molecule for an activity on neutrophil differentiation is provided, the method comprising i) providing the candidate molecule, ii) causing the candidate molecule to contact unipotent neutrophil progenitor cells to determine the activity of the candidate molecule on the cell differentiation of said unipotent cells into neutrophils, and iii) receiving information conveying the activity of the candidate molecule on the cell differentiation of said unipotent cells into neutrophils.

In certain aspects of the present invention, a method for treatment or prevention of neutropenia in a subject is provided, the method comprising administering to the subject an effective amount of a purified unipotent neutrophil progenitor cell population. In alternative embodiments, said progenitor cells are autologous cells to the subject.

In certain aspects of the present invention, use of an effective amount of a purified unipotent neutrophil progenitor cell population for treatment or prevention of neutropenia in a subject is provided. In alternative embodiments, said progenitor cells are autologous cells to the subject.

In certain aspects of the present invention, use of an effective amount of a purified unipotent neutrophil progenitor cell population in the manufacture of a medicament for treatment or prevention of neutropenia in a subject is provided. In alternative embodiments, said progenitor cells are autologous cells to the subject.

In certain other aspects of the present invention, a method of inhibiting or preventing tumor growth in a subject is provided, the method comprising inhibiting differentiation of unipotent neutrophil progenitor cells into neutrophil cells in said subject.

In certain other aspects of the present invention, use of an inhibitor for inhibiting or preventing tumor growth in a subject is provided, where the inhibitor inhibits differentiation of unipotent neutrophil progenitor cells into neutrophil cells in the subject.

In certain other aspects of the present invention, use of an inhibitor in the manufacture of a medicament for inhibiting or preventing tumor growth in a subject is provided, where the inhibitor inhibits differentiation of unipotent neutrophil progenitor cells into neutrophil cells in the subject.

In certain aspects of the present invention, a pharmaceutical composition comprising isolated unipotent neutrophil progenitor cells and a pharmaceutically acceptable carrier is provided, wherein said progenitor cells are modified so as to have modified gene expression, modified cell function, or to include a ribonucleic acid interference (RNAi) causing molecule, or a conjugated therapeutic agent. In some aspects, the cells are genetically modified by CRISPR-cas9, lentivirus transduction or RNAi.

In some aspects of the present invention, the biological sample described herein includes blood or a cell fraction thereof. In still other aspects, said biological sample includes blood, spleen, tumor tissue or bone marrow, or a cell fraction thereof.

In certain aspects of the present invention, said reference level described herein is derived from a cohort of at least 20 reference individuals without disease condition. In certain alternative aspects, said reference level is derived from a sample from the subject, the sample being provided prior to or after a treatment performed to treat the subject.

In alternative aspects of the present invention, said subject is afflicted with neutropenia. In other aspects, said neutropenia is caused by a cancer.

In certain aspects of the present invention, the progenitor cells have at least the phenotype CD45+, CD41−, CD127 (IL-7Rα)−, CD19−, CD3−, CD161 (NK1.1)−, CD169 (Siglec 1)−, CD11c−, Siglec 8−, FcεRIα− and CD115 (CSF-1R)−. In other aspects, the progenitor cells have at least the phenotype CD161−, CD34+, CD38+, CD115−, Siglec8−, FcεRIα− and CD114+.

In some aspects of the present invention, the progenitor cells have at least the phenotype CD45+, CD235ab−, CD41−, CD127 (IL-7Rα)−, CD19−, CD3−, CD4−, CD161 (NK1.1)−, CD56−, CD169 (Siglec 1)−, CD64−, CD11c−, HLA-DR−, CD86−, CD123−, CD7−, CD10−, CD366−, CD90−, Siglec 8−, FcεRIα−, CD115 (CSF-1R)−, CD34+, CD38+, CD45RA+, CD66b+, CD16b+, CD15+, CD114+, CD14int, CD162int, and CD62Lint.

In some aspects of the present invention, the progenitor cells have at least the phenotype hSiglec 8−, hFcεRIα−, hCD3−, hCD7−, hCD10−, hCD11c−, hCD19−, hCD41−, hCD56−, hCD90 (Thy1)−, hCD123 (IL-3Rα)−, hCD125 (IL-5Rα)−, hCD127 (IL-7Rα)−, hCD161−, hCD169−, hCD235a−, hCD66b+, hCD117 (c-Kit)+, hCD38+, and hCD34+ (e.g. Subset A as described herein).

In some aspects of the present invention, the progenitor cells have at least the phenotype hSiglec 8−, hFcεRIα−, hCD3−, hCD7−, hCD10−, hCD11c−, hCD19−, hCD41−, hCD56−, hCD90 (Thy1)−, hCD123 (IL-3Rα)−, hCD125 (IL-5Rα)−, hCD127 (IL-7Rα)−, hCD161−, hCD169−, hCD235a−, hCD34−, hCD66b+, hCD117 (c-Kit)+, and hCD38+ (e.g. Subset B as described herein).

In certain aspects of the present invention, said subject is human. In alternative aspects, said subject is a mouse.

In some aspects of the present invention, the progenitor cells have at least the phenotype CD161−, CD117(c-Kit)+, Ly6A/E−, CD16/32+, CD115−, SiglecF−, FcεRIα− and Ly6G−/lo. In other aspects, the progenitor cells have at least the phenotype CD45+, Ter119−, CD41−, CD127 (IL-7Rα)−, CD19- or B220−, CD3−, TCRβ−, CD161 (NK1.1)−, CD335 (NKp46)−, CD169 (Siglec 1)−, F4/80−, CD11c−, MHCII−, CD117 (c-kit)+/int, Ly6A/E (Sca1)−, Siglec F (Siglec 8)−, FcεRIα−, CD115 (CSF-1R)−, Ly6C−/int, CD16/32 (FcγRIII/II)+, and Ly6G−/lo.

In certain aspects of the present invention, the progenitor cells have at least the phenotype CD41−, CD127(IL-7Rα)−, CD3−, CD19−, CD161(NK1.1)−, CD169(Siglec 1)−, CD11c−, Siglec F, FcERIα−, CD115(CSF-1R)−, Ly6A/E(Sca1)−, Ly6G−, CD162(PSGL-1) lo, CD48 lo, Ly6C lo, and CD117(c-Kit)+, CD16/32(FcγRIII/II)+, Ly6B+ and CD11a(LFA1α)+ (e.g. Cluster #C1 as described herein).

In certain aspects of the present invention, the progenitor cells have at least the phenotype CD41−, CD127(IL-7Rα)−, CD3, CD19−, CD161(NK1.1)−, CD169(Siglec 1)−, CD11c−, Siglec F−, FcERIα−, CD115(CSF-1R)−, Ly6A/E(Sca1)−, CD117(c-Kit)+, CD16/32(FcγRIII/II)+, Ly6B, CD11a(LFA1α)+, and Ly6G+ (e.g. Cluster #C2 as described herein).

In certain aspects of the present invention, a kit for sorting unipotent neutrophil progenitor cells from a biological sample is provided, the kit comprising detecting agents for CD161, CD34, CD38, CD115, Siglec8, FcεRIα and CD114. In alternative aspects, the kit comprises detecting agents for CD45, CD41, CD127 (IL-7Rα), CD19, CD3, CD161 (NK1.1), CD169 (Siglec 1), CD11c, Siglec 8, FcεRIα and CD115 (CSF-1R). In other aspects, the kit comprises detecting agents for CD45, CD235ab, CD41, CD127 (IL-7Rα), CD19, CD3, CD4, CD161 (NK1.1), CD56, CD169 (Siglec 1), CD64, CD11c, HLA-DR, CD86, CD123, CD7, CD10, CD366, CD90, Siglec 8, FcεRIα, CD115 (CSF-1R), CD34, CD38, CD45RA, CD66b, CD16b, CD15, CD114, CD14, CD162, and CD62L. In other aspects, the kit comprises detecting agents for hSiglec 8, hFcεRIα, hCD3, hCD7, hCD10, hCD11c, hCD19, hCD41, hCD56, hCD90 (Thy1), hCD123 (IL-3Rα), hCD125 (IL-5Rα), hCD127 (IL-7Rα), hCD161, hCD169, hCD235a, hCD66b, hCD117 (c-Kit), hCD38, and hCD34.

In certain aspects of the present invention, the use of a kit for sorting unipotent neutrophil progenitor cells from a biological sample is provided, where the kit is as defined herein.

All features of embodiments which are described in this disclosure and are not mutually exclusive can be combined with one another. Elements of one embodiment can be utilized in the other embodiments without further mention. Other aspects and features of the present invention will become apparent to those ordinarily skilled in the art upon review of the following description of specific embodiments in conjunction with the accompanying Figures.

### BRIEF DESCRIPTION OF THE DRAWINGS

A detailed description of specific embodiments is provided herein below with reference to the accompanying drawings in which:

FIG. 1A shows a non-limiting embodiment of an automated single-cell analysis of Lin⁻ CD117⁺Ly6A/E⁻ cells, identifying a distinct novel neutrophil progenitor population. Mass Cytometry (CyTOF) was used to define a largest Cluster #C of the 5 subsets in Lin⁻ CD117⁺Ly6A/E⁻ cells from murine BM. BM cells isolated from C57BL/6J donors were stained with the antibody panel shown in FIG. 8. B cells (B220⁺), T cells (TCRβ⁺), Macrophage cocktail (CD169⁺, F4/80⁺), Erythroid/lymphoid cocktail (CD41⁺, Ter119⁺, CD127⁺), DCs (CD11c⁺, MHCII⁺), NK cells (CD335⁺, CD161⁺), were excluded from single live CD45⁺ cells for Lin⁻ cells. ViSNE maps of Lin⁻ CD117⁺ Ly6A/E⁻ cells are shown as dot overlays to display the 5 automated clusters (#A-E). Ly6G expression pattern is shown on viSNE map of Lin⁻ CD117⁺Ly6A/E⁻ cells as spectrum of dots. The expression patterns of the indicated markers are shown as histogram overlays of each cluster. Results are representative of two independent experiments (n=6 mice each).

FIG. 1B left, shows a non-limiting embodiment of two PhenoGraph meta-clusters presenting two distinct populations (1, 2) in Cluster #C. FIG. 1B, right, shows a non-limiting embodiment of the expression profile of Ly6G, Ly6C, and Ly6B for randomly selected cells in each cluster visualized on the first component of a nonlinear dimensionality reduction isomap (the regression black line estimated using the generalized linear model is added for each marker).

FIG. 1C shows a non-limiting embodiment of the FACS gating strategy for Cluster #C. Manually gated Cluster #C is back gated to automated viSNE map for validation.

FIG. 2A shows a non-limiting embodiment of ScRNA-Seq analysis of Cluster #C, revealing two major subpopulations #C1 and #C2. 20,000 Cluster #C cells were sorted from healthy wild-type mice BM for scRNA-Seq assay (3 biological triplicates, 2 technical replicates). Left, tSNE 2D plots, obtained applying Seurat scRNA-Seq analysis R Package for the scRNA-Seq data, showing two main clusters corresponding to subsets of Cluster #C (n=16268 cells; #C1, 2149 cells and #C2, 14089 cells. Right, Violin plots show the single cell expression pattern of indicated transcripts for #C1 and #C2 clusters. Shapes represent the distribution of cells based on their expression values (y-axis). Grey scale represents the mean expression. Heatmap shows top 40 differentially expressed genes in each cluster. Log 2 Fold Change of each gene expression is relative to the entire dataset.

FIG. 2B shows a non-limiting embodiment of the FACS gating strategy for Cluster #A and D, #B, #C1, #C2, and #E. Manually gated clusters are back gated to automated viSNE map for validation.

FIG. 3A shows, as non-limiting embodiment, Cluster #C1 cells express little to very low levels Ly6G and #C2 express low levels of Ly6G compared to BM Neutrophils by mass cytometry. Results are representative of two independent experiments (n=6 mice each).

FIG. 3B shows a non-limiting embodiment of FACS sorting of cell subsets from healthy wild-type mice. 3-dimensional reconstructions of nuclear architecture in Cluster #C1, Cluster #C2, BM neutrophils (BM Neuts), and Blood neutrophils (Blood Neuts). Bar: 10 μm.

FIG. 3C shows a non-limiting embodiment of Ki67 localization within the nuclei in Cluster #C1 and #C2 detected via confocal microscopy. #C1, #C2, BM Neuts, and Blood Neuts were sorted and stained with antibodies to Ki67 and DNA was labeled with Hoechst. IgG stained cells served as a negative control. Bar: 5 μm.

FIG. 3D shows a non-limiting embodiment of sorting of Cluster #C1, #B (CD115⁺), #A, D, and #E cells from wild-type mice and diluted to single-cell suspension. Single cell of each cluster were cultured in methylcellulose-base medium. Numbers of colonies generated from the indicated progenitors were counted at day 10 of the culture. Contingency plot shows mean value of six independent experiments (each contains 3 biological triplicates).

FIG. 4A shows a non-limiting embodiment of sorting of Cluster #C1, #C2, #B (CD115⁺), #A, D, and #E cells from CD45.2 donors and the adoptive transfer into irradiated wild-type CD45.1 recipient mice. Each recipient group includes 25 mice. After the transfer, peripheral blood was collected for flow cytometry of CD45. 2⁺ cells from 5 recipients of each group at days (D) 5, 7, 12, 14, 28 (D5, D7, D12, D14, D28), respectively. CD45.2⁺ cells were evaluated for the donor cell-derived monocytes (CD115⁺), neutrophils (Ly6G⁺), eosinophils (Siglec F⁺), and basophils (FcERIα⁺). N=5 mice for each time point in each group.

FIG. 4B shows a non-limiting embodiment of the appearance of neutrophils and monocytes via representative plots showing the appearance in each recipient group at the time points indicated. Results are representative of two independent experiments.

FIG. 4C shows a non-limiting embodiment of the percentage of neutrophils in CD45.2⁺ cells from each group in FIG. 4B. Solid bars represent neutrophils; open bars represent other CD45.2⁺ cells.

FIG. 4D shows a non-limiting embodiment of the time points that CD45.2⁺ cells appear in peripheral blood of each recipient group in FIG. 4B.

FIG. 5A shows a non-limiting embodiment of Cluster #C1 cells increased in BM with tumor and promoting tumor growth in vivo. 5×10⁵ B16F10 melanoma cells were SubQ injected into the rear flank of wild-type recipient mice for primary tumor growth. The frequency of Cluster #E, #B (CD115⁺), and #C1 were detected in BM from tumor-bearing mice at 14 d post-injection (open bars) or their healthy counterparts (solid bars). Results are representative of 3 independent experiments. N=5 mice in each group. Error bars indicate the s.d. value.

FIG. 5B shows a non-limiting embodiment of (left) Cluster #E, #B (CD115⁺), and #C1 being sorted from the same CD45.2 wild type donors and adoptively transferred into sub-lethally irradiated congenic CD45.1 recipients. The next day, 3×10⁵ B16F10 melanoma cancer cells were SubQ injected into each recipient mouse. (Right) The tumor size in each recipient was measured at 12 d post-injection. Results are representative of 2 independent experiments. N=5 mice in each group. Error bars indicate the s.d. value.

FIG. 6A shows a non-limiting embodiment of flow cytometry analysis of healthy human BM, showing a heterogeneous Lin⁻hCD66b⁺hCD117⁺ fraction. Dump antibody cocktail contains: hSiglec 8, hFcεRIα, hCD3, hCD7, hCD10, hCD11c, hCD19, hCD41, hCD56, hCD90 (Thy1), hCD123 (IL-3Rα), hCD125 (IL-5Rα), hCD127 (IL-7Rα), hCD161, hCD169, and hCD235a (Glycophorin A). N=3 healthy donors.

FIG. 6B shows a non-limiting embodiment of ScRNA-Seq analysis of Lin⁻ hCD66b⁺hCD117⁺ cells, revealing two major subpopulations Subset A and Subset B. 20,000 cells were FACS sorted from healthy human BM for scRNA-Seq. Heatmap shows top 40 differentially expressed genes in each cluster. Log 2 Fold Change of each gene expression is relative to the entire dataset. 2 biological triplicates, 2 technical replicates.

FIG. 6C shows a non-limiting embodiment automated viSNE analysis of this Lin⁻ hCD66b⁺hCD117⁺ fraction, revealing 2 major clusters. The two clusters express different levels of hCD15, hCD38, and hCD16.

FIG. 6D shows a non-limiting embodiment of the FACS sorting of Subset A and Subset B from healthy human BM based on hCD34 expression. Confocal microscopy was used to detect Ki67 localization within the nuclei in hCD34⁺ Subset A and hCD34⁻ Subset B using antibodies to Ki67 and Hoechst. IgG stained cells served as negative control. Bar: 5 μm.

FIG. 7A shows a non-limiting embodiment of hNeP production of neutrophils in NSG-SGM3 (NSG-M3) mice. hCD34⁺ Subset A and hCD34⁻ Subset B were FACS sorted from healthy human BM. The two subsets were adoptively transferred into NSG-M3 recipient mice. Each recipient mouse received 25,000 donor human progenitor cells. After the transfer, peripheral blood was collected from each recipient via saphenous vein for flow cytometry on Day (D) 5, 7, 14, 28 (D5, D7, D14, D28), respectively.

FIG. 7B shows a non-limiting embodiment of representative plots showing the appearance of monocytes (hCD86⁺hCD66b), neutrophils (hCD86⁻ hSiglec 8⁻hCD66b⁺), eosinophils (hSiglec hCD66b⁺), and lymphocytes (hLy⁺) in each recipient group at the time points indicated. hLy antibody cocktail contains hCD3, hCD19, and hCD56. N=10 mice for each time point.

FIG. 7C shows a non-limiting embodiment showing the experiment procedure (Left). hCD34⁺ Subset A, hCD34⁻ Subset B, and human cMoP were FACS sorted from healthy human BM. The 3 populations were adoptively transferred into NSG-M3 recipient mice. Blank control group received only PBS for adoptive transfer. The next day, 1×10⁶ 143B human osteosarcoma cells were SubQ injected into each recipient mouse. (Right), the tumor size in each recipient was measured at 10 d post-injection. N=5 mice in each group. Error bars indicate the s.d. value.

FIG. 7D shows a non-limiting embodiment of detection of hNeP frequency by flow cytometry in peripheral blood collected from healthy donors (n=3) and melanoma patients (n=3). Error bars indicate the s.d. value.

FIG. 8 shows the antibody table used to perform CyTOF mass cytometry on healthy mouse bone marrow.

FIG. 9A shows a non-limiting embodiment of the FACS gating strategy for Cluster #B (CD115⁺) fraction. Manually gated Cluster #B (CD115⁺) fraction is back gated to automated viSNE map for validation.

FIG. 9B shows a non-limiting embodiment of Cluster #C cells increased in periphery with tumor. B16F10 melanoma cells were SubQ injected into the rear flank of wild-type recipient mice for primary tumor growth. The frequency of Cluster #C cells in blood and spleen from tumor-bearing mice at 14 d post-injection (open bars) or their healthy counterparts (solid bars) were detected by flow cytometry. Results are representative of 3 independent experiments. N=5 mice in each group. Error bars indicate the s.d. value.

FIG. 10A shows a non-limiting embodiment of Flow cytometry analysis of human BM aspirate, showing live CD45⁺ cells contain a hCD66b⁺hCD34⁺ fraction and a hCD66b⁺hCD117⁺ fraction.

FIG. 10B shows a non-limiting embodiment of FMO controls for hNeP gating.

FIG. 11A shows a non-limiting embodiment of a positive staining control group, comprising Human peripheral blood collected from healthy donors (n=3) and stained with the same antibody cocktail as in FIG. 7B. Human derived cells were evaluated for monocytes (hCD86⁺hCD66b), neutrophils (hCD86⁻ hSiglec 8⁻hCD66b⁺), eosinophils (hSiglec 8⁺ hCD66b⁺), and lymphocytes (hLy⁺). hLy antibody cocktail contains hCD3, hCD19, and hCD56.

FIG. 11B shows a non-limiting embodiment of representative plots showing the appearance of monocytes (hCD86⁺hCD66b), neutrophils (hCD86⁻ hSiglec 8⁻hCD66b⁺), eosinophils (hSiglec 8⁺ hCD66b⁺), and lymphocytes (hLy⁺) in each recipient group at the time points indicated. hLy antibody cocktail contains hCD3, hCD19, and hCD56. N=10 mice for each time point.

FIG. 12A shows a non-limiting embodiment of CyTOF analysis of neutrophil precursors. Previously identified neutrophil precursor (termed K.NeuP here) was gated as described by (Kim et al., 2017) with the CyTOF dataset in FIG. 1. Side-by-side viSNE analysis of this population and the Lin⁻CD117⁺Ly6A/E⁻ population revealed heterogeneity in the K.NeuP population.

FIG. 12B shows a non-limiting embodiment of Cluster #C1 and #C2 as gated with the gating strategy shown in FIG. 2B and overlaid with the Lin⁻CD117⁺Ly6A/E⁻ viSNE map in FIG. 12A.

FIG. 13A shows a non-limiting embodiment of a schematic of adoptive transfer of NePs in a tumor model and resulting FACS data from tumor. Donor BM NePs are recruited by tumor into circulation and generate CD11b+Ly6G+ progenies. NePs were sorted from CD45.2 wild type donors and were adoptively transferred into sub-lethally irradiated congenic CD45.1/2 recipients. The next day, 5×10⁵ B16F10 cells were SubQ injected into each recipient mouse. At D8 after the adoptive transfer, the blood and tumor mass were harvested from recipients. Donor-NeP and progeny (CD45.2+) were evaluated using flow cytometry.

FIG. 13B shows a non-limiting embodiment of results obtained for an experimental assay in which the adoptive transfer of NePs promotes tumor growth. Left, NePs, MonPs, and LSK⁺ HSPCs were sorted from wild type donors. Equal numbers of MonPs and NePs were mixed with LSK⁺ HSPCs, respectively. MonPs+LSK⁺ or NePs+LSK⁺ were adoptively transferred into sub-lethally irradiated recipients, respectively (n=15 each). Right top graph, FACS data of number of monocytes following administration of either MonPs+LSK⁺ or NePs+LSK. Right bottom graph, the sizes of tumor in each mouse were measured at the 7th day of growth. Error bars indicate the standard deviation (s.d.) of triplicates. Statistical significance was determined using the unpaired Student t-test, **P<0.01.

FIG. 14A shows a non-limiting embodiment of manual gating strategy of LSK⁻ HSPC for flow cytometry is defined with the methods described for mass cytometry. The name of the parent cell population is indicated on the top or at the top left of each 2-dimensional plot. The spectrum expression pattern for the marker indicated at the right bottom of each 2-dimensional plot is shown with high expression corresponding with the top of the spectrum and low expression corresponding with the bottom of the spectrum.

FIG. 14B shows a non-limiting embodiment of a CD117 FMO stained BM sample that is used as negative control for accurate CD117⁺ gate. The name of the parent cell population is indicated on the top or at the top left of each 2-dimensional plot. The spectrum expression pattern for the marker indicated at the right bottom of each 2-dimensional plot is shown with high expression corresponding with the top of the spectrum and low expression corresponding with the bottom of the spectrum.

FIG. 14C shows a non-limiting embodiment of a viSNE automated mapping of LSK⁻ HSPC with flow cytometry data. The name of the parent cell population is indicated on the top or at the top left of each 2-dimensional plot. The spectrum expression pattern for the marker indicated at the right bottom of each 2-dimensional plot is shown with high expression corresponding with the top of the spectrum and low expression corresponding with the bottom of the spectrum.

FIG. 15A shows a non-limiting embodiment of intranuclear expression of Ki67 in NePs from blood, spleen, and tumor mass of tumor-bearing mice (14 d of tumor) compared to whole blood cells measured by flow cytometry. NePs in circulation of tumor-bearing mice are proliferative. Similar results were obtained in four independent experiments.

FIG. 15B shows a non-limiting embodiment of live CD45⁺ leukocytes from tumor mass and blood of tumor-bearing mice (14 d of tumor) and healthy counterparts were evaluated for CD11b⁺Ly6G⁺ subset frequency by flow cytometry. Similar results were obtained in four independent experiments.

FIG. 15C shows a non-limiting embodiment of intranuclear expression of Ki67 in donor-derived NePs in tumor mass from experimental group in FIG. 13A measured by flow cytometry. Donor BM NePs that are recruited to tumor mass are proliferative.

FIG. 15D shows a non-limiting embodiment of FACS data of adoptively transferred NePs in the blood. Donor BM NePs are recruited by tumor into circulation and generate CD11b⁺Ly6G⁺ progenies. Blood were harvested from the experiment group in FIG. 13A. The donor-NeP and its progeny (CD45.2⁺) were evaluated using flow cytometry.

In the drawings, embodiments are illustrated by way of example. It is to be expressly understood that the description and drawings are only for the purpose of illustrating certain embodiments and are an aid for understanding. They are not intended to be a definition of the limits of the invention.

### DETAILED DESCRIPTION

Illustrative embodiments of the disclosure will now be more particularly described. While the making and using of various embodiments of the present disclosure are discussed in detail below, it should be appreciated that the present disclosure provides many applicable inventive concepts that can be embodied in a wide variety of specific contexts. The specific embodiments discussed herein are merely illustrative of specific ways to make and use the invention and do not delimit the scope of the invention.

While modern techniques such as multi-color flow cytometry techniques have enabled the discovery of several unipotent or oligopotent progenitors in hematopoiesis, including the Eosinophil Progenitor (EoP) (10, 11), Basophil/Mast Cell Progenitors (B/MCP) (12, 13), and multiple Monocyte Progenitors (here termed MonPs), which consist of the Monocyte/Dendritic Cell (DC) Progenitor (MDP), the common Monocyte Progenitor (cMoP), and the recently discovered Segregated-nucleus-containing atypical Monocyte Progenitor (SatMP) (14-17), however, it is unknown if unipotent neutrophil progenitor cells exist.

Although the existence of bonafide neutrophil precursors has been suggested in several studies (18-20), the unipotency of these precursors to strictly produce only neutrophils has not been shown (21).

Recently, high-dimensional mass cytometry (also known as cytometry by time-of-flight, CyTOF), which combines the advantages of both flow cytometry and mass spectrometry by utilizing antibodies conjugated to metal isotopes, has become a powerful tool to investigate the hematopoietic system (22-24). Using mass cytometry, high heterogeneity of hematopoietic progenitors within the BM has been demonstrated (22, 23). While, mass cytometry analysis of the mouse BM with a myeloid-selective panel of surface markers revealed a cell subset with morphology highly related to reported neutrophil precursors (22), however, the developmental potential of this subset was not evaluated.

In the present specification, the inventors describe an enriched or purified preparation of novel neutrophil progenitor population, methods of making a preparation of such neutrophil progenitor population, and methods of using same.

In the present specification, the inventors describe the discovery of a new, very early-stage, committed unipotent neutrophil progenitor (NeP) that is present in mouse and human bone marrow. The inventors have found that both the mouse and human NeP promoted primary tumor growth in vivo in established cancer models. Further, the presence of the human NeP (hNeP) in the blood of patients with recently diagnosed melanoma was identified, showing that this hNeP is released from the bone marrow in patients with cancer, and can be readily identified in human blood.

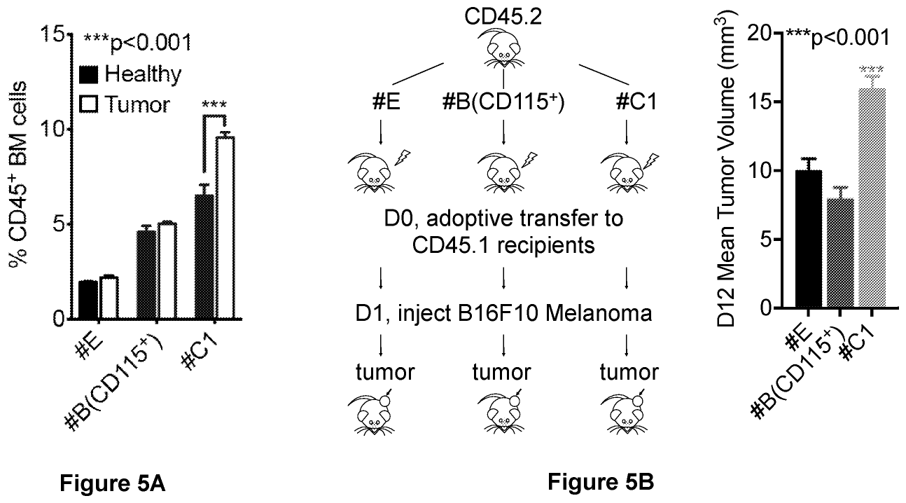

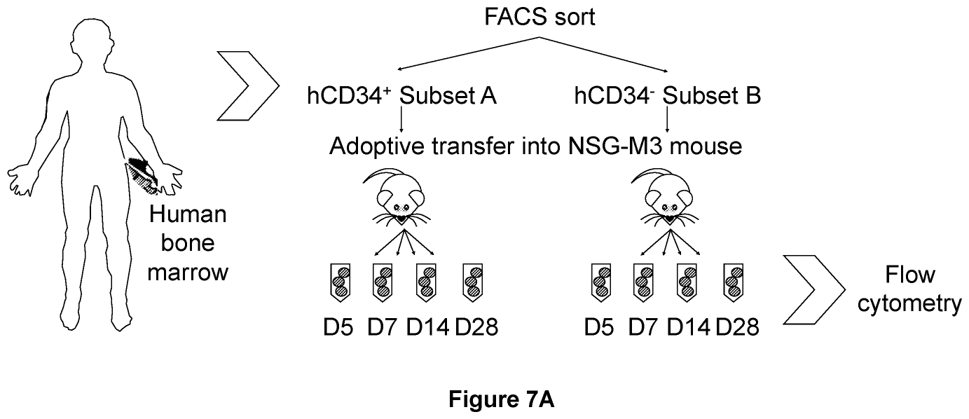

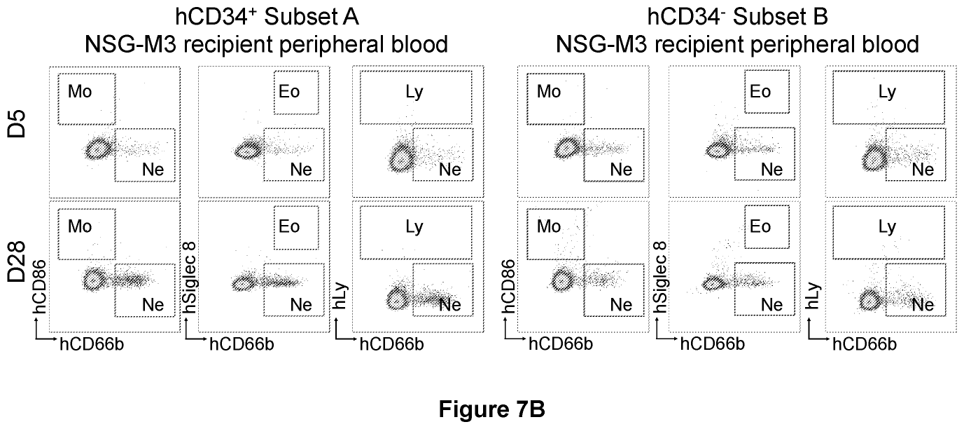

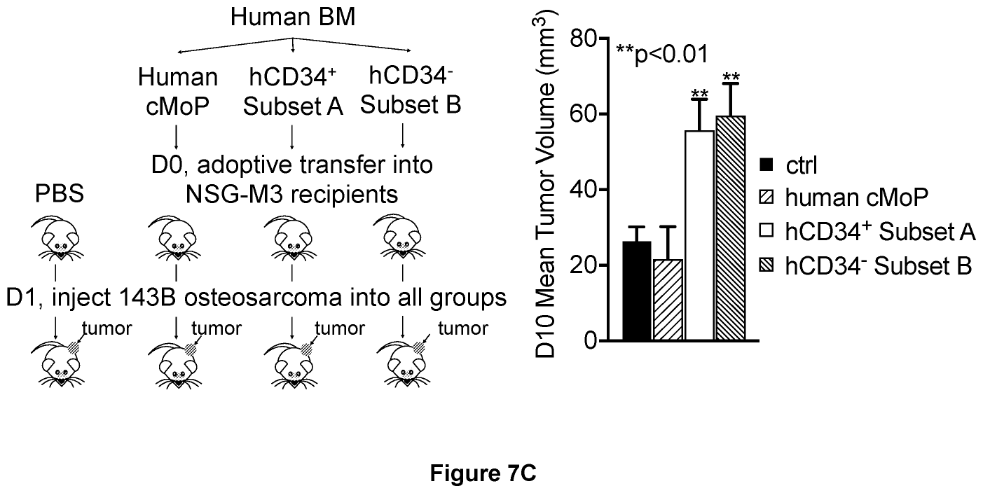

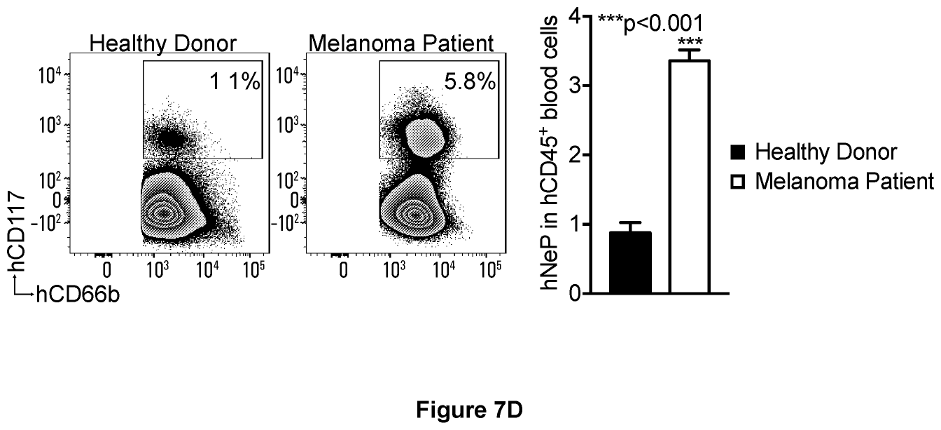

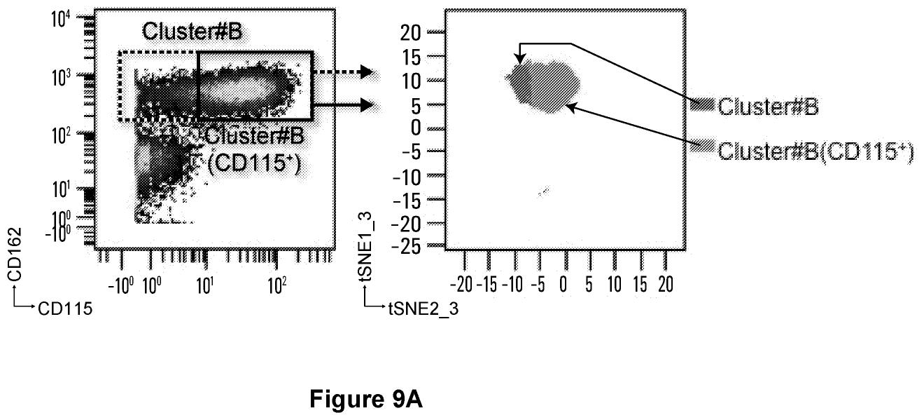

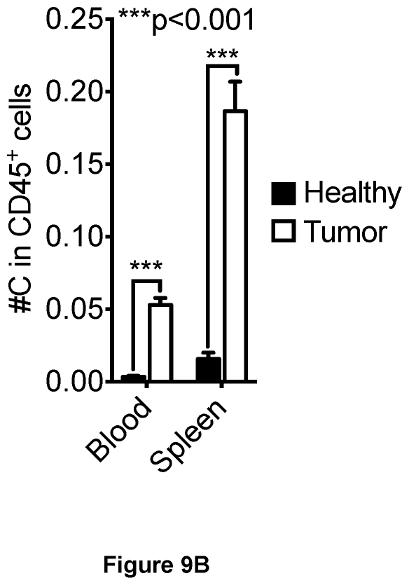

Importantly, a tumor-promoting role for this new early-stage neutrophil progenitor was discovered in both mice and humans. In tumor-bearing mice, frequencies of this NeP are increased in bone marrow, showing aberrant myelopoiesis in response to tumor growth (FIG. 5A). These results are consistent with previous studies that show that the tumor reprograms GMP to cause increased production of tumor-associated neutrophils (Casbon et al., 2015). It was discovered that tumor-induced myelopoiesis is specific for NeP in mouse BM (FIG. 5A) and not other myeloid progenitors. Further, when adoptively transferred into recipient mice, the NeP significantly promoted melanoma tumor growth compared to other myeloid progenitors and was also found in the periphery, showing egress from the bone marrow in the setting of cancer (FIG. 9B). Similar tumor-promoting effects of hNeP were detected in human tumorigenesis using a NSG humanized mouse model. After adoptive transfer, hNeP significantly promoted osteosarcoma tumor growth in NSG mice compared to other myeloid progenitors (FIG. 7C). A 5-6 fold increase of hNeP in the blood of patients diagnosed with melanoma was observed. This result is consistent with the observation of increased NeP in in mouse periphery in response to tumor growth (FIG. 9b), and, without being bound to a particular theory, demonstrates that this hNeP can be used as a biomarker for early cancer detection.

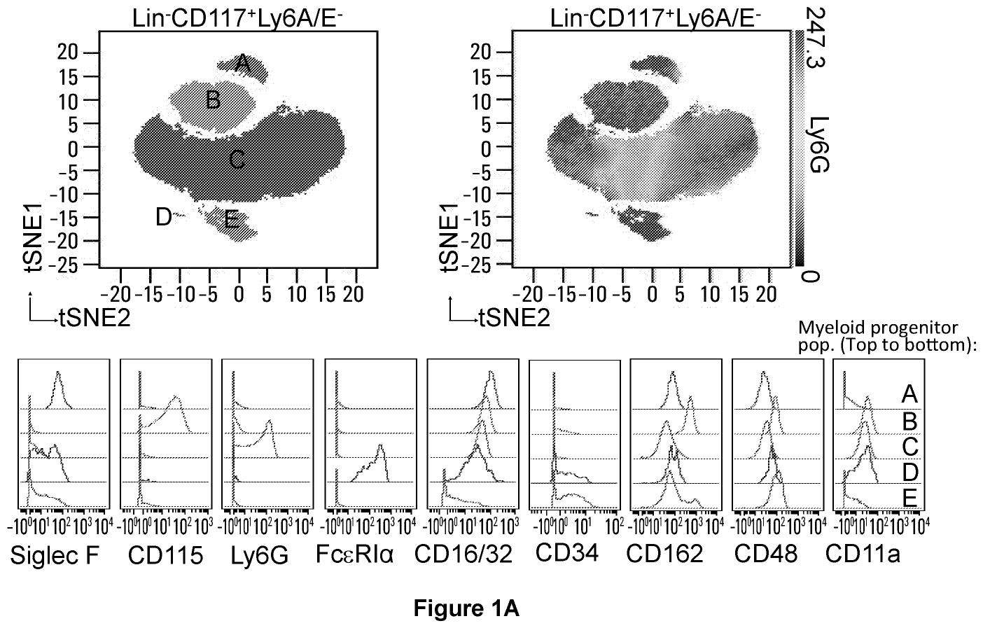

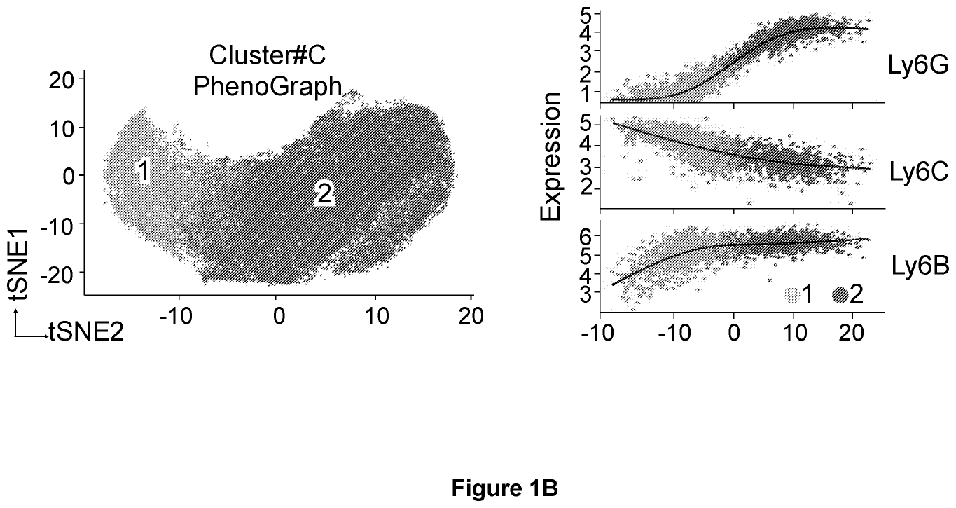

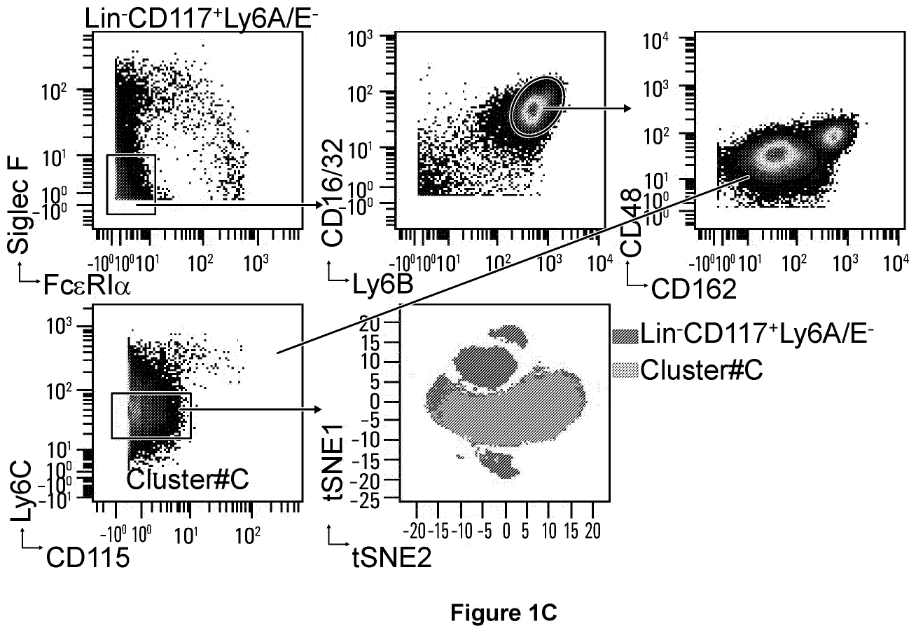

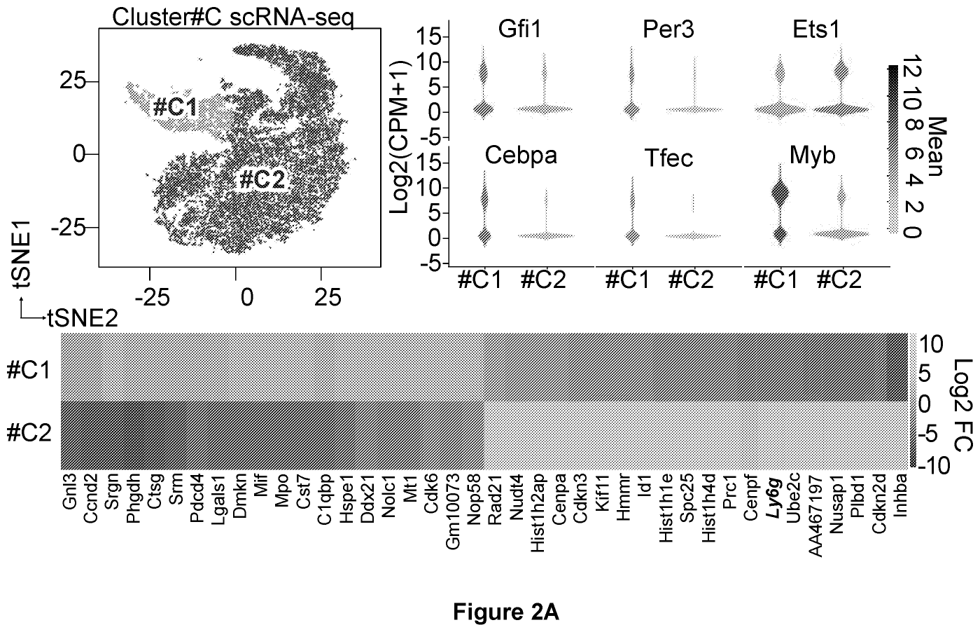

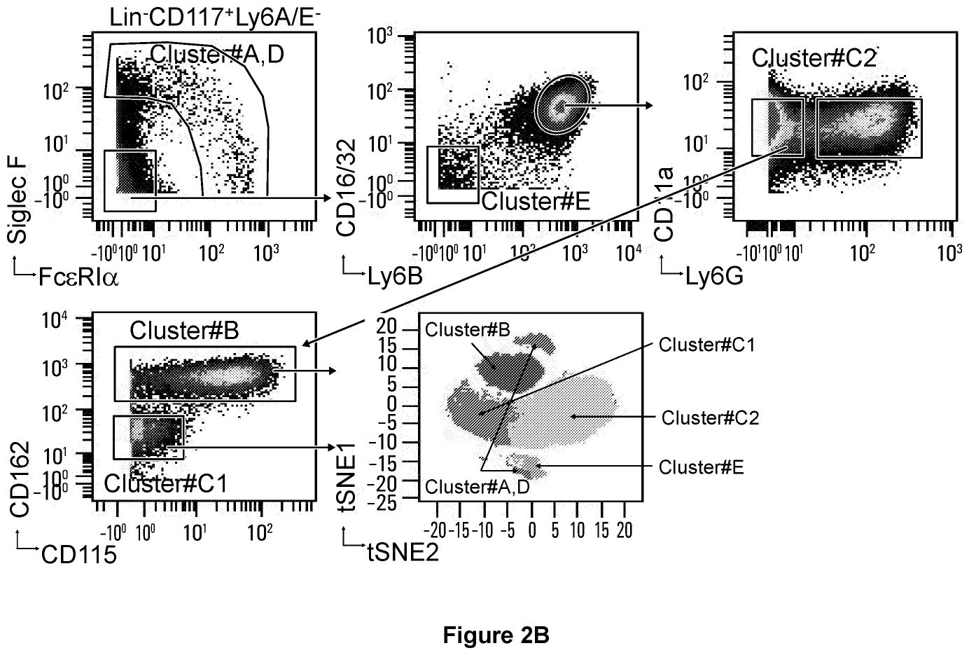

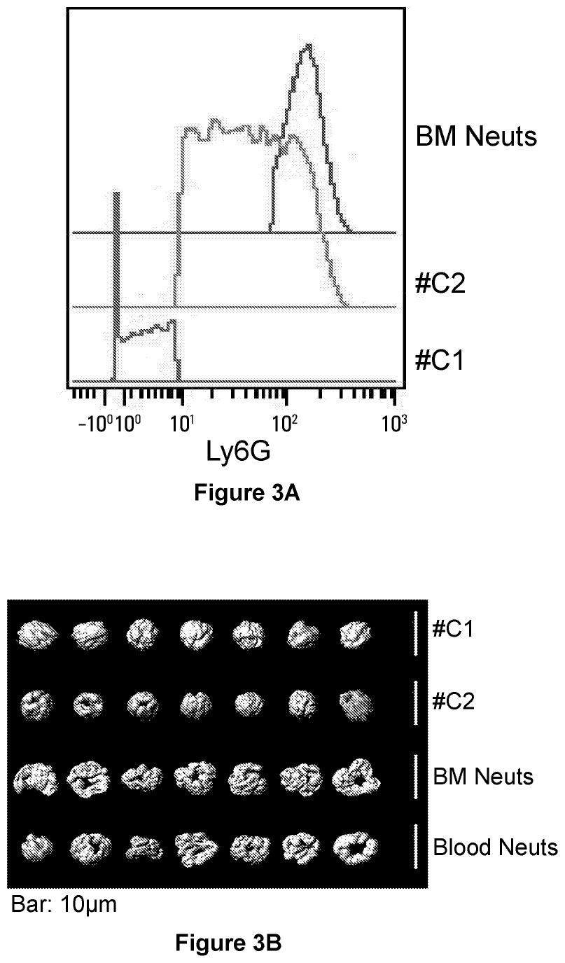

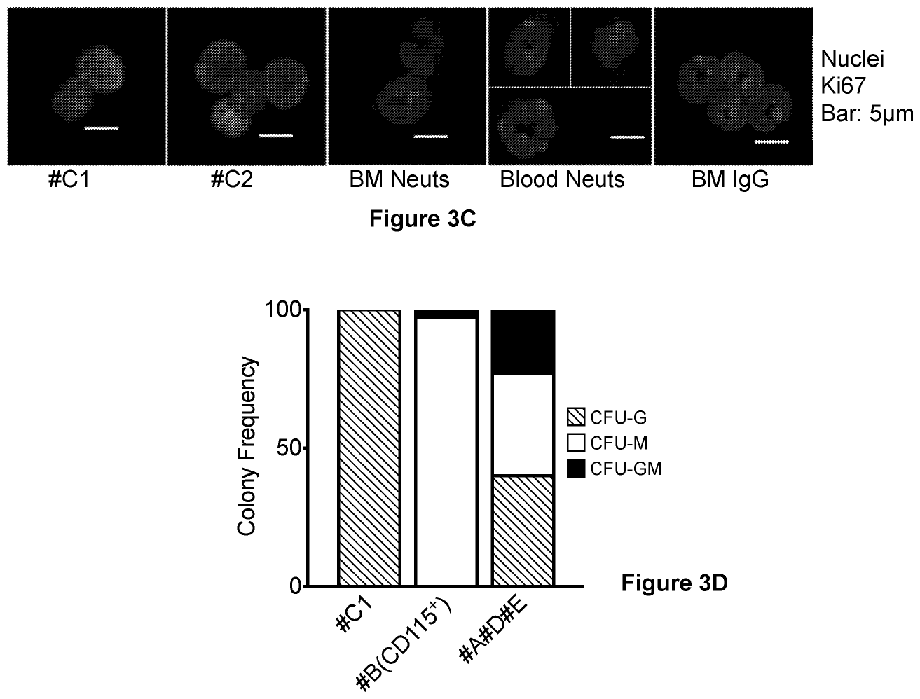

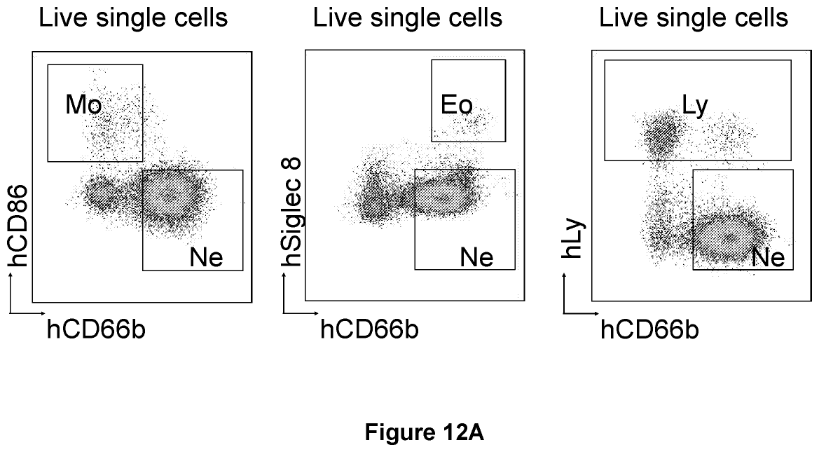

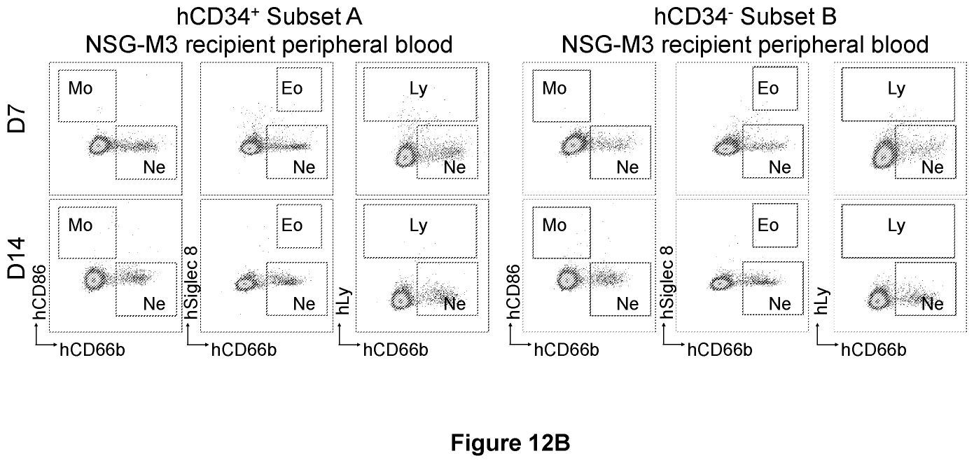

The earliest committed neutrophil progenitor has remained elusive for decades. Most studies have focused on murine hematopoiesis. In this regard, the classic model of hematopoiesis shows that LSK⁺ (Lin⁻CD117⁺Ly6A/E⁺CD127) HSPCs give rise to CLP (Lin⁻ CD117^{lo} Ly6A/E⁺CD1271 for lymphopoiesis and to the Lin⁻CD117⁺Ly6A/E⁻CD127⁻ HSPCs for myelopoiesis (Weissman et al., 2001). However, further examination of the Lin⁻CD117⁺Ly6A/E⁻ HSPC fraction by mass cytometry showed 5 committed myeloid progenitors (FIG. 1A). Cluster #C in FIG. 1A showed low to moderate expression of Ly6G, providing a neutrophil lineage potential for cells found within this cluster. This cluster was not identified in earlier hematopoiesis studies as the neutrophil marker Ly6G was routinely excluded from flow cytometry panels at that time. ScRNA-Seq analysis of this Ly6G-containing Cluster #C further revealed 2 populations: an early-stage progenitor (#C1) with stem-cell morphology and little Ly6G expression and a late-stage precursor (#C2) that expressed low levels of Ly6G with morphological features similar to transient neutrophil precursors and immature neutrophils (FIGS. 2A-2B and 3A-3D) (Satake et al., 2012; Sturge et al., 2015; Yáñez et al., 2015). Recently, a late-stage neutrophil precursor was identified in bone marrow of mice (Kim et al., 2017). The inventors located a late-stage neutrophil precursor in the bone marrow of mice (K.NeuP) on a viSNE map of Lin⁻CD117⁺Ly6A/E⁻ HSPCs (FIG. 12A). It was discovered that this K.NeuP population was highly heterogeneous and possibly contaminated with other myeloid progenitors. The inventors were able to generate via mass cytometry data a stringent flow cytometry gating strategy (FIG. 2B) that allowed for the complete purification, with no contamination from other myeloid lineages, both #C1 (NeP) and #C2 cells (late-stage precursors and immature neutrophils) (FIG. 12B) in order to demonstrate their neutrophil unipotency.

In sum, using mass cytometry the inventors have identified a novel, new, early-stage committed unipotent neutrophil progenitor that is present in both mouse and human bone marrow. This discovery provides new therapeutic and pharmaceutical targets for neutrophil-related diseases or treatment outcomes that are associated with chronic inflammation. For example, neutropenia leads to high susceptibility to infections and is often associated as a by-product of cancer treatments (Lyman et al., 2014). Without being bound to a particular theory, targeting hNeP may rescue patients from undesirable neutropenia. In addition, the inventors' observation of increased hNeP in blood of melanoma patients provides avenues for early detection for cancer diagnosis as a biomarker. As this hNeP also displays tumor-promoting effects, without being bound to a particular theory, this hNeP itself could be an immune-oncology target.

### Progenitor Population

The progenitor population of the present disclosure is also referred to hereinafter as Neutrophil Progenitors (NePs). The progenitor population of the present disclosure includes progenitor cells that give rise, upon differentiation, to only neutrophils. Such ability can be tested in vitro and/or in vivo with the herein described methods or with methods that are readily available to the person of skill in the art. Accordingly, the NePs of the present disclosure are hereinafter also referred to as unipotent neutrophil progenitor cells.

In one embodiment, the progenitor population of the present disclosure includes a cell population having at least the phenotype CD115−, Siglec8− and FcERIα−.

In one embodiment, the progenitor population of the present disclosure includes a cell population having at least the phenotype CD45+, CD41−, CD127 (IL-7Rα)−, CD19−, CD3−, CD161 (NK1.1)−, CD169 (Siglec 1)−, CD11c−, Siglec 8−, FcERIα− and CD115 (CSF-1R)−.

In one embodiment, the progenitor population of the present disclosure includes a mouse cell population having at least the phenotype CD117(c-Kit)+, CD16/32+, CD115−, SiglecF−, FcERIα−.

In one embodiment, the progenitor population of the present disclosure includes a mouse cell population having at least the phenotype CD161−, CD117(c-Kit)+, Ly6A/E−, CD16/32+, CD115−, SiglecF−, FcERIα− and Ly6G−/lo.

In one embodiment, the progenitor population of the present disclosure includes a mouse cell population having at least the phenotype CD45+, Ter119−, CD41−, CD127 (IL-7Rα)−, CD19- or B220−, CD3−, TCRβ−, CD161 (NK1.1)−, CD335 (NKp46)−, CD169 (Siglec 1)−, F4/80−, CD11c−, MHCII−, CD117 (c-kit)+/int, Ly6A/E (Sca1)−, Siglec F (Siglec 8)−, FcERIα−, CD115 (CSF-1R)−, Ly6C−/int, CD16/32 (FcγRIII/II)+, and Ly6G−/lo.

In one embodiment, the progenitor population of the present disclosure includes a mouse cell population having at least the phenotype CD41−, CD127(IL-7Rα)−, CD3−, CD19−, CD161(NK1.1)−, CD169(Siglec 1)−, CD11c−, Siglec F, FcERIα−, CD115(CSF-1R)−, Ly6A/E(Sca1)−, Ly6G−, CD162(PSGL-1) lo, CD48 lo, Ly6C lo, and CD117(c-Kit)+, CD16/32(FcγRIII/II)+, Ly6B+ and CD11a(LFA1α)+.

In one embodiment, the progenitor population of the present disclosure includes a mouse cell population having at least the phenotype CD41−, CD127(IL-7Rα)−, CD3, CD19−, CD161(NK1.1)−, CD169(Siglec 1)−, CD11c−, Siglec F−, FcERIα−, CD115(CSF-1R)−, Ly6A/E(Sca1)−, CD117(c-Kit)+, CD16/32(FcγRIII/II)+, Ly6B, CD11a(LFA1α)+, and Ly6G+.

In one embodiment, the progenitor population of the present disclosure includes a human cell population having at least the phenotype CD34+, CD38+, CD115−, Siglec8− and FcERIα−.

In one embodiment, the progenitor population of the present disclosure includes a human cell population having at least the phenotype CD161−, CD34+, CD38+, CD115−, Siglec8−, FcERIα− and CD114+.

In one embodiment, the progenitor population of the present disclosure includes a human cell population having at least the phenotype CD45+, CD235ab−, CD41−, CD127 (IL-7Rα)−, CD19-, CD3−, CD4−, CD161 (NK1.1)−, CD56−, CD169 (Siglec 1)−, CD64−, CD11c−, HLA-DR−, CD86−, CD123−, CD7−, CD10−, CD366−, CD90−, Siglec 8−, FcERIα−, CD115 (CSF-1R)−, CD34+, CD38+, CD45RA+, CD66b+, CD16b+, CD15+, CD114+, CD14int, CD162int, and CD62Lint.

In one embodiment, the progenitor population of the present disclosure includes a human cell population having at least the phenotype hSiglec 8−, hFcεRIα−, hCD3−, hCD7−, hCD10−, hCD11c−, hCD19−, hCD41−, hCD56−, hCD90 (Thy1)−, hCD123 (IL-3Rα)−, hCD125 (IL-5Rα)−, hCD127 (IL-7Rα)−, hCD161−, hCD169−, hCD235a−, hCD66b+, hCD117 (c-Kit)+, hCD38+, and hCD34+.

In one embodiment, the progenitor population of the present disclosure includes a human cell population having at least the phenotype hSiglec 8−, hFcεRIα−, hCD3−, hCD7−, hCD10−, hCD11c−, hCD19−, hCD41−, hCD56−, hCD90 (Thy1)−, hCD123 (IL-3Rα)−, hCD125 (IL-5Rα)−, hCD127 (IL-7Rα)−, hCD161−, hCD169−, hCD235a−, hCD34−, hCD66b+, hCD117 (c-Kit)+, and hCD38+.

In the present disclosure, “−” refers to negative expression, “+” refers to positive expression, the term “lo” refers to negative or low expression levels, “int” refers to intermediate expression levels and “hi” refers to high expression levels.

In one embodiment, the progenitor population of the present disclosure includes a cell population that further expresses Lymphocyte antigen 6 complex locus G6D (hereinafter, a cell of further phenotype Ly6G⁺). In another embodiment, the progenitor population of the present disclosure includes a cell population that does not express Lymphocyte antigen 6 complex locus G6D (hereinafter, a cell of further phenotype Ly6G). In yet another embodiment, the progenitor population of the present disclosure includes a first cell population of further phenotype Ly6G⁺ and a second cell population of further phenotype Ly6G⁻.

In one embodiment, the progenitor population of the present disclosure includes a first cell population of further phenotype Ly6G⁺ and a second cell population of further phenotype Ly6G⁻ in a ratio Ly6G⁺/Ly6G⁻ which is selected based on a desired neutrophil differentiation kinetics when the progenitor population is introduced in a subject. The person of skill can, thus, prepare a composition comprising the progenitor population of the present disclosure where the composition includes a first cell population of further phenotype Ly6G⁺ and a second cell population of further phenotype Ly6G⁻ in a ratio Ly6G⁺/Ly6G⁻ which is selected based on a desired neutrophil differentiation kinetics when the progenitor population is introduced in a subject. Such composition, thus, does not exist in nature and is functionally different from a comparison composition which is extracted (e.g., cell sorted) from a natural biological sample since this composition will have different neutrophil differentiation kinetics when the progenitor population is introduced in a subject, where such kinetics are purposively selected by the person of skill by specifically designing the composition to have a given ratio Ly6G⁺/Ly6G⁻.

In one embodiment, the progenitor population of the present disclosure may include a cell population with cells that have been modified, for example but without being limited thereto, so as to have modified gene expression, modified cell function or to include a ribonucleic acid interference (RNAi)-causing molecule, or to have a conjugated therapeutic agent.

In one embodiment, the progenitor population of the present disclosure may include a cell population with cells that have been genetically modified by CRISPR-cas system (such as CRISPR/Cas9), Cre-lox recombination system, gene knock-down, gene knock-out, lentivirus transduction or RNAi-causing molecule.

For example, the progenitor population of the present disclosure may include a cell population with cells that have been further modified so as to include an RNAi-causing molecule such as a short hairpin RNA (shRNA), a small interfering RNA (siRNA), a micro RNA (miRNA), or a plasmid DNA for expressing the shRNA, siRNA or miRNA. RNAi-causing molecules are well known in the art. For example, the person of skill will readily understand that miRNA are small (e.g., 18-25 nucleotides in length), noncoding RNAs that influence gene regulatory networks by post-transcriptional regulation of specific messenger RNA (mRNA) targets via specific base-pairing interactions. This ability of microRNAs to inhibit the production of their target proteins results in the regulation of many types of cellular activities, such as cell-fate determination, apoptosis, differentiation, and oncogenesis.

The person of skill will readily recognized that the progenitor population of the present disclosure may be modified in vitro and/or in vivo, with techniques that are readily available to the person of skill, so as to obtain cells having the desired characteristic.

### Method of Preparation of the Progenitor Population

In one embodiment, the progenitor population of the present disclosure may be extracted from a biological sample using a cell sorting technique. For example, the cell sorting technique may include flow-cytometry-based cell sorting, magnetic cell sorting, and/or antibody panning.

In one embodiment, the cell sorting technique may be carried out in a device adapted to separate or quantify cells on the basis of detecting agent(s) binding to specific cell markers in the progenitor population of the present disclosure. The detecting agent(s) may further include cell sorting agent(s), such as a chromophore or a metal. When the detecting agent includes a chromophore, the device may be, for example, a fluorescence-activated cell sorting (FACS) device. The specific markers of the progenitor population of the present disclosure may be intracellular markers and/or cell surface markers. For example, the detecting agent may include antibodies, which may further include a cell sorting agent as described above.

The cell measurements may be carried out, for example, by immunoassays including, but not limited to, western blots, immunohistochemistry, immunocytochemistry, in situ hybridization, radioimmunoassays, ELISA (enzyme linked immunosorbent assay), “sandwich” immunoassays, immunoprecipitation assays, precipitin reactions, gel diffusion precipitin reactions, immunodiffusion assays, agglutination assays, complement-fixation assays, immune-radiometric assays, fluorescent immunoassays, immunofluorescence, or flow cytometry.

In one embodiment, the progenitor population of the present disclosure may be extracted from a biological sample using at least one of the gating strategies which are provided in Example 1.

As used herein, the progenitor population of the present disclosure may be extracted from a sample which includes bone marrow, tumor tissue, blood or spleen, or a cell fraction thereof.

In one embodiment, the present disclosure relates to a kit for cell sorting the progenitor cells of the present disclosure. For example, such kit may include a combination of detecting agents for any combination of the previously described markers.

For example, the kit may include a combination of detecting agents for markers such as at least CD115, Siglec8 and FcERIα; or at least CD45, CD41, CD127 (IL-7Rα), CD19, CD3, CD161 (NK1.1), CD169 (Siglec 1), CD11c, Siglec 8, FcERIα and CD115 (CSF-1R); or at least CD117(c-Kit), CD16/32, CD115, Siglec F, FcERIα; or at least CD34, CD38, CD115, Siglec 8 and FcERIα.

In another example, such kit may include a combination of detecting agents for markers such as at least CD161, CD117(c-Kit), Ly6A/E, CD16/32, CD115, SiglecF, FcERIα and Ly6G; or at least CD45, Ter119, CD41, CD127 (IL-7Rα), CD19 or B220, CD3, TCRβ, CD161 (NK1.1), CD335 (NKp46), CD169 (Siglec 1), F4/80, CD11c, MHCII, CD117 (c-kit), Ly6A/E (Sca1), Siglec F (Siglec 8), FcERIα, CD115 (CSF-1R), Ly6C, CD16/32 (FcγRIII/II), and Ly6G.

In another example, such kit may include a combination of detecting agents for markers such as at least CD41, CD127(IL-7Rα), CD3, CD19, CD161(NK1.1), CD169(Siglec 1), CD11c, Siglec F, FcERIα, CD115(CSF-1R), Ly6A/E(Sca1), Ly6G, CD162(PSGL-1), CD48, Ly6C, and CD117(c-Kit), CD16/32(FcγRIII/II), Ly6B and CD11a(LFA1α).

In another example, such kit may include a combination of detecting agents for markers such as at least CD41, CD127(IL-7Rα), CD3, CD19, CD161(NK1.1), CD169(Siglec 1), CD11c, Siglec F, FcERIα, CD115(CSF-1R), Ly6A/E(Sca1), CD117(c-Kit), CD16/32(FcγRIII/II), Ly6B, CD11a(LFA1α), and Ly6G.

In another example, such kit may include a combination of detecting agents for markers such as at least CD161, CD34, CD38, CD115, Siglec8, FcERIα and CD114; or at least CD45, CD235ab, CD41, CD127 (IL-7Rα), CD19, CD3, CD4, CD161 (NK1.1), CD56, CD169 (Siglec 1), CD64, CD11c, HLA-DR, CD86, CD123, CD7, CD10, CD366, CD90, Siglec 8, FcERIα, CD115 (CSF-1R), CD34, CD38, CD45RA, CD66b, CD16b, CD15, CD114, CD14, CD162, and CD62L.

In another example, such kit may include a combination of detecting agents for markers such as at least hSiglec 8, hFcεRIα, hCD3, hCD7, hCD10, hCD11c, hCD19, hCD41, hCD56, hCD90 (Thy1), hCD123 (IL-3Rα), hCD125 (IL-5Rα), hCD127 (IL-7Rα), hCD161, hCD169, hCD235a, hCD66b, hCD117 (c-Kit), hCD38, and hCD34.

The person of skill will readily recognize that various permutation of detecting agent may be included in such kits so as to obtain the desired result.

### Applications for Using the Progenitor Population

The present disclosure further describes methods which make use of the progenitor population of the present disclosure to obtain a desired result, which may be for example, but without being limited thereto, therapeutic and/or prophylactic, or which may further provide information on neutrophil biology in health and/or disease, or which may assist in evaluating the effectiveness of a given treatment, and the like.

In one embodiment, the present disclosure describes a method for treatment of a subject. The method may include activation or inhibition of the progenitor population of the present disclosure to differentiate into neutrophils. In other words, the person of skill may implement steps to target the progenitor population of the present disclosure. Such method may have therapeutic and/or prophylactic desired results. The activation or inhibition may occur in vitro, in which case, the resulting activated or inhibited progenitor population of the present disclosure can then be administered to the subject in order to obtain the desired result. Alternatively or additionally, the activation or inhibition may occur in vivo with the administration of a suitable activation or inhibition compound to the subject.

For example, activation of the progenitor population of the present disclosure may include contacting the progenitor population with a suitable compound targeting transcription factors such as Gfi1, Snai1, or KLF5. If the progenitor population of the present disclosure includes a human cell population, then the suitable activating compound may target CD114 (G-CSFR). In certain embodiments, activation of the progenitor population of the present disclosure may include administering a drug suitable for treatment of neutropenia (e.g., G-CSF, Docetaxel). Inhibition of the progenitor population of the present disclosure may include contacting the progenitor population with a suitable compound targeting transcription factors such as Gata1, IRF8, or KLF4. In certain embodiment, inhibition of the progenitor population of the present disclosure may include administering a drug suitable for treatment of neutrophilia (e.g., Imatinib).

In one embodiment, the suitable compound may be a ribonucleic acid interference inducing (RNAi) molecule, a small molecule, an antibody, a protein, a peptide, a ligand mimetic, and the like. The person of skill will readily understand what compound may be suitable to obtain the desired effect.

This method of treatment can be used conjunctly with an assessment step as described below.

In a first variant, the above method of treatment may further include an assessment step whereby one determines the levels of the progenitor population of the present disclosure which are present in the subject pre- and/or post-treatment. In order to do so, the person of skill may implement additional steps whereby the levels of the progenitor population of the present disclosure are determined in a biological sample of the subject. In certain non-limiting embodiments, the biological sample here includes blood, spleen, tumor tissue, or bone marrow, or a cell fraction thereof. Such additional steps may comprise processing the biological sample being suspected of including the progenitor population of the present disclosure to determine the concentration or activation level thereof. In one embodiment, such additional steps may make use of the cell sorting techniques described earlier to extract the progenitor population of the present disclosure from the biological sample.

In a second variant, the above method of treatment may further include an assessment step whereby one determines the levels of the neutrophil cells which are present in the subject pre- and/or post-treatment. In order to do, the person of skill may implement additional steps whereby the levels of the neutrophil cells are determined in another biological sample of the subject. In one non-limiting embodiment, the biological sample here includes blood or a cell fraction thereof. Such additional steps may comprise processing the biological sample being suspected of including the neutrophil cells to determine the level thereof. In one non-limiting embodiment, such additional steps may make use of cell sorting techniques, as described elsewhere in the present document or that are readily available to the person of skill in the art. In another variant, the person of skill may make use of readily available detecting agents that selectively recognize markers present on the neutrophil cells and which can be detected/quantified so as to indirectly determine the concentration level of neutrophils.

In a third variant, the above method of treatment may include a combination of the first and second variant.

As discussed elsewhere in the present document, the level which is determined from the biological sample can be compared to a reference level. In certain embodiments, the reference level can be derived from a sample of at least 20 reference individuals without condition (in other words that are not afflicted by the condition of the tested subject), or at least 30, or at least 40, or at least 50, or at least 60, or at least 100 reference individuals without condition. Alternatively or additionally, the reference level can be derived from levels determined in the subject pre and/or post treatment.

In one practical implementation, such variants can, thus, serve to determine the effectiveness of a given treatment by providing clinical information pertaining to a subject's neutrophil levels and/or NePs levels in pre and/or post treatment phase. For example, the person of skill can monitor the effectiveness of a method for treatment or prevention of cancer, neutropenia or related conditions. Such monitoring can be performed by implementing at least one of the herein described variants.

In one embodiment, neutropenia can be caused by a cancer. For example, a cancer selected from colon carcinomas, pancreatic cancer, breast cancer, lung carcinoma, prostate cancer, metastatic renal cell carcinoma (RCC), mammary carcinoma, lung cancer, thymoma, fibrosarcoma, and myeloid sarcoma.

In another embodiment, neutropenia can be caused by chemotherapy, severe microbial infection (such as Hepatitis, HIV/AIDS, malaria or Salmonella), sepsis (overwhelming blood infection that depletes neutrophils faster than they can be produced), Kostmann's syndrome, myelokathexis or other congenital disorders, leukemia, myelodysplastic syndromes, autoimmune disorders such as Rheumatoid arthritis, neonates with growth disorders or those born to mothers with preeclampsia or hypertension, or transplant.

The present disclosure also describes a method for evaluating a cancer in a subject. Generally speaking, this method includes determining a concentration or activation level of the neutrophil progenitor population of the present disclosure in a biological sample of the subject, which is suspected of including the neutrophil progenitor population of the present disclosure. The biological sample here may include blood, spleen, tumor tissue, bone marrow or a cell fraction thereof. In one embodiment, the biological sample may include blood or a cell fraction thereof. The method further includes comparing the concentration or activation level to a reference level. At least based on such comparison, the person of skill can then determine the likelihood that the subject has or does not have cancer. Indeed, the data presented in the present document provide factual basis for the person of skill to reasonably expect that the concentration or activation level of the neutrophil progenitor population of the present disclosure is indicative of the presence of cancer in a subject.

In a variant of such method for evaluating a cancer in a subject, the person of skill can also determine the response or resistance to cancer treatment in a subject undergoing cancer treatment. Indeed, following treatment, the person of skill can determine the concentration or activation level of the neutrophil progenitor population of the present disclosure which will be indicative of the progression of the cancer and accordingly, will provide information as to the response or resistance to cancer treatment in the subject undergoing cancer treatment. In other words, when comparing the concentration or activation level to a reference level, the person of skill can evaluate the response or resistance to the treatment based on at least the comparison.

In another variant, of such method for evaluating a cancer in a subject, the cancer may cause neutropenia. In such variant, the person of skill can also determine the response or resistance to a treatment for a condition associated with neutropenia in the subject undergoing the treatment. Indeed, following treatment, the person of skill can determine the concentration or activation level of the neutrophil progenitor population of the present disclosure which will be indicative of the neutrophil differentiation capability of the subject. In other words, when comparing the concentration or activation level to a reference level, the person of skill can evaluate the response or resistance to the treatment based on at least the comparison.

The present disclosure also describes a method for reducing risk of cancer progression or cancer relapse in a subject. The method includes determining a concentration or activation level of the neutrophil progenitor population of the present disclosure in a biological sample of the subject, which is suspected of including the neutrophil progenitor population of the present disclosure. The biological sample here may include blood, spleen, tumor tissue, bone marrow or a cell fraction thereof. In one embodiment, the biological sample may include blood or a cell fraction thereof. The method further includes comparing the concentration or activation level to a reference level. At least based on such comparison, the person of skill can then selectively administer a cancer therapeutic agent so as to reduce risk of cancer progression or cancer relapse in the subject.

In one embodiment, the present disclosure also describes a method for reducing risk of a condition associated with neutropenia in the subject. The method includes determining a concentration or activation level of the neutrophil progenitor population of the present disclosure in a biological sample of the subject, which is suspected of including the neutrophil progenitor population of the present disclosure. The biological sample here may include blood, spleen, tumor tissue, bone marrow or a cell fraction thereof. In one embodiment, the biological sample may include blood or a cell fraction thereof. The method further includes comparing the concentration or activation level to a reference level. At least based on such comparison, the person of skill can then selectively administer a therapeutic agent so as to reduce risk of the condition neutropenia in the subject.

The comparison step includes using a reference level. The reference level can be derived from a sample of at least 20 reference individuals without condition (in other words that are not afflicted by the condition of the tested subject), or at least 30, or at least 40, or at least 50, or at least 60, or at least 100 reference individuals without condition. Alternatively or additionally, the reference level can be derived from levels determined in the subject pre and/or post treatment.

In one embodiment, the present disclosure also describes a method for screening a candidate molecule for an activity on cell differentiation of the neutrophil progenitor population of the present disclosure into neutrophils. The method includes contacting the neutrophil progenitor population of the present disclosure with the candidate molecule and determining the activity of the candidate molecule on the cell differentiation of the neutrophil progenitor population of the present disclosure into neutrophils.

In one embodiment, the present disclosure also describes a method for treatment or prevention of neutropenia in a subject. The method includes administering to the subject an effective amount of a purified preparation of the neutrophil progenitor population of the present disclosure. Such administration can be used in conjunction with the assessment steps described earlier in this document, for example, to monitor the effectiveness of the treatment.

In one embodiment, the neutrophil progenitor population of the present disclosure which is administered to the subject includes progenitor cells that are autologous (cells from the subject being administered), allogeneic (cells from another individual), or syngenic (genetically identical, or sufficiently identical and immunologically compatible as to allow for transplantation), to the subject.

### Definitions

Unless otherwise defined, all technical and scientific terms used herein have the same meaning as commonly understood by a person of ordinary skill in the art to which the present invention pertains. As used herein, and unless stated otherwise or required otherwise by context, each of the following terms shall have the definition set forth below.

In one embodiment, the methods described herein make use of the measured levels of the progenitor population of the present disclosure to detect surges or declines in cell numbers as predictive measures. As used herein, a “surge” indicates a statistically significant increase in the level of relevant cells, typically from one measurement to one or more later measurements. In other instances, an increase in the level of relevant cells can be determined from one measure in a subject of interest relative to control (e.g., a value or a range of values for normal, i.e., healthy, individuals). Surges may be a two-fold increase in cell levels (i.e., a doubling of cell counts), a three-fold increase in cell levels (i.e., a tripling of cell numbers), a four-fold increase in cell levels (i.e., an increase by four times the number of cells in a previous measurement), or a five-fold or greater increase. In addition to the marked increase described as a surge, lesser increases in the levels of relevant cells may also have relevance to the methods of the present disclosure. Increases in cell levels may be described in terms of percentages. Surges may also be described in terms of percentages. For example, a surge or increase may be an increase in cell levels of 10%, 20%, 30%, 40%, 50%, 60%, 70%, 80%, 90%, or 100% or more. A “decline” indicates a decrease from one measurement to one or more later measurements. A decline may be a 5%, 10%, 15%, 20%, 25%, 30%, 35%, 40%, 45%, 50%, 55%, 60%, 65%, 70%, 75%, 80%, or 85% or greater decrease in cell levels from one measurement to one or more later measurements. In other instances, a decrease in the level of relevant cells can be determined from one measure in a subject of interest relative to control (e.g., a value or a range of values for normal, i.e., healthy, individuals).

In one embodiment, the surges or declines in cell numbers can be determined based on a comparison with a reference level derived from samples of at least 20 reference individuals without condition, a non-patient population. The surges or declines in cell numbers in a sample can also refer to a level that is elevated in comparison to the level of the cell numbers reached upon treatment, for example with an anti-cancer compound.

In one embodiment, the term “cancer” refers to a class of diseases in which a group of cells display uncontrolled growth, invasion, and metastasis. The term is meant to include, but not limited to, a cancer of the breast, respiratory tract, brain, reproductive organs, digestive tract, urinary tract, eye, liver, skin, head and neck, thyroid, and parathyroid. The cancer may be a solid tumor, a non-solid tumor, or a distant metastasis of a tumor. Some specific examples of cancers include, but are not limited to, leukemia; lymphomas; multiple myelomas; bone and connective tissue sarcomas; brain tumors; breast cancer; adrenal cancer; thyroid cancer; pancreatic cancer; pituitary cancers; eye cancers; vaginal cancers; cervical cancers; uterine cancers; ovarian cancers; esophageal cancers; stomach cancers; colon cancers; rectal cancers; gastric cancers; liver cancers; bladder cancers; gallbladder cancers; cholangiocarcinoma; lung cancers; testicular cancers; prostate cancers; penile cancers; oral cancers; basal cancers; salivary gland cancers; pharynx cancers; skin cancers; kidney cancers; and Wilms' tumor. Examples of solid tumors include solid tumors of the breast, prostate, colon, pancreas, lung, gastric system, bladder, and bone/connective tissue. When making reference to neutropenia in particular, the cancer can be selected from colon carcinomas, pancreatic cancer, breast cancer, lung carcinoma, prostate cancer, metastatic RCC, mammary carcinoma, lung cancer, thymoma, fibrosarcoma, and myeloid sarcoma.

As used herein, “relapse” or “recurrence” may include the appearance of at least one new tumor lesions in a subject who previously had cancer but has had no overt evidence of cancer as a result of surgery and/or therapy until relapse. Such recurrence of cancer cells can be local, occurring in the same area as one or more previous tumor lesions, or distant, occurring in a previously lesion-free area, such as lymph nodes or other areas of the body.

As used herein, “response to treatment” may include complete response and partial response to treatment. A “complete response” (CR), in certain embodiments relating to e.g. cancer, is typically understood to include the disappearance of all target lesions and non-target lesions and normalization of tumor marker levels, whereas in other embodiments relating to e.g. neutropenia, is typically understood as the complete normalization of neutrophil levels in the subject. A “partial response” (PR), in certain embodiments relating to cancer, is typically understood to include an at least 30% decrease in the sum of the diameters of target lesions, whereas in other embodiments relating to neutropenia, is typically understood as a relative increase of neutrophil levels in a subject suffering from neutropenia of at least 30%. Generally speaking, in the context of embodiments relating to e.g. cancer, “response to treatment” may include an at least 30%-100% decrease in the sum of the diameters of target lesions, or disappearance of all target lesions and non-target lesions and normalization of tumor marker levels. Generally speaking, in the context of embodiments relating to e.g. neutropenia, “response to treatment” may include an at least 30%-100% increase in neutrophil levels. “Progression” or “progressive disease” (PD), in certain embodiments relating to e.g. cancer, is typically understood to include an at least 20% increase in the sum of the diameters of target lesions, progression (increase in size) of any existing non-target lesions, and is also typically determined upon appearance of at least one new lesion. Non-CR/non-PD, in certain embodiments relating to e.g. cancer, is typically understood to include the persistence of one or more non-target lesions and/or maintenance of above-normal tumor marker levels. “Stable disease” (SD) is typically understood to include an insufficient increase to qualify for PD, but an insufficient decrease to qualify for PR. While the concepts of CR, PR, PD, and SD have been discussed in the context of cancer and neutropenia, the person of skill will readily understand that these concepts may also apply to other disease/conditions, which are associated with aberrant neutrophil levels.

As used herein, the terms “treatment”, “treating”, and the like, may include amelioration or elimination of a developed disease or condition once it has been established or alleviation of the characteristic symptoms of such disease or condition. As used herein, these terms may also encompass, depending on the condition of the subject, preventing the onset of a disease or condition or of symptoms associated with the disease or condition, including for example reducing the severity of the disease or condition or symptoms associated therewith prior to affliction with the disease or condition. Such prevention or reduction prior to affliction may refer, in the context of cancer, to administration of at least one cancer therapeutic compound to a subject that is not at the time of administration afflicted with the disease or condition. “Preventing” may also encompass preventing the recurrence or relapse of a previously existing disease or condition or of symptoms associated therewith, for instance after a period of improvement.

The subject or patient can be any mammal, including a human. In particular, in the context of cancer, the subject can be a mammal who previously had cancer but appears to have recovered as a result of surgery and/or therapy, or who presently has cancer and is undergoing cancer therapy, or has completed a cancer therapeutic regime, or has received no cancer therapy.

As used herein, the terms “therapeutically effective amount” and “effective amount” are used interchangeably to refer to an amount of a composition of the disclosure that is sufficient to result in the prevention of the development, recurrence, or onset of a disease or condition. For example, in certain embodiments e.g. cancer, these terms refer to an amount of a composition of the invention that is sufficient to result in the prevention of the development, recurrence, or onset of cancer stem cells or cancer and one or more symptoms thereof, to enhance or improve the prophylactic effect(s) of another therapy, reduce the severity and duration of cancer, ameliorate one or more symptoms of cancer, prevent the advancement of cancer, cause regression of cancer, and/or enhance or improve the therapeutic effect(s) of additional anticancer treatment(s). For example, in certain embodiments e.g. neutropenia, these terms refer to an amount of a composition of the disclosure that is sufficient to result in the prevention of the development, recurrence, or onset of neutropenia or one or more symptoms thereof, to enhance or improve the prophylactic effect(s) of another therapy, reduce the severity and duration of neutropenia, ameliorate one or more symptoms of neutropenia, prevent the advancement of neutropenia (further decrease of neutrophil levels), and/or enhance or improve the therapeutic effect(s) of additional anti-neutropenia treatment(s).

A therapeutically effective amount can be administered to a patient in one or more doses sufficient to palliate, ameliorate, stabilize, reverse or slow the progression of the disease, or otherwise reduce the pathological consequences of the disease, or reduce the symptoms of the disease. The amelioration or reduction need not be permanent, but may be for a period of time ranging from at least one hour, at least one day, or at least one week or more. The effective amount is generally determined by the physician on a case-by-case basis and is within the skill of one in the art. Several factors are typically taken into account when determining an appropriate dosage to achieve an effective amount. These factors include age, sex and weight of the patient, the condition being treated, the severity of the condition, as well as the route of administration, dosage form and regimen and the desired result.

In one non-limiting embodiment, the biological sample from the subject which is suspected of including neutrophil cells includes blood or a cell fraction thereof.

In one non-limiting embodiment, the biological sample from the subject which is suspected of including the progenitor population of the present disclosure includes blood, spleen, tumor tissue, bone marrow or a cell fraction thereof.

As used herein, a “cell fraction” of a biological sample may be obtained using routine clinical cell fractionation techniques, such as gentle centrifugation, e.g., centrifugation at about 300-800×g for about five to about ten minutes, or fractionated by other standard methods.

In one non-limiting embodiment, the herein described sample can be obtained by any known technique, for example by drawing, by non-invasive techniques, or from sample collections or banks, etc.

In one non-limiting embodiment, the present disclosure provides a kit which includes reagents that may be useful for implementing at least some of the herein described methods. The herein described kit may include at least one detecting agent which is “packaged”. As used herein, the term “packaged” can refer to the use of a solid matrix or material such as glass, plastic, paper, fiber, foil and the like, capable of holding within fixed limits the at least one detection reagent. Thus, in one non-limiting embodiment, the kit may include the at least one detecting agent “packaged” in a glass vial used to contain microgram or milligram quantities of the at least one detecting agent. In another non-limiting embodiment, the kit may include the at least one detecting agent “packaged” in a microtiter plate well to which microgram quantities of the at least one detecting agent has been operatively affixed. In another non-limiting embodiment, the kit may include the at least one detecting agent coated on microparticles entrapped within a porous membrane or embedded in a test strip or dipstick, etc. In another non-limiting embodiment, the kit may include the at least one detecting agent directly coated onto a membrane, test strip or dipstick, etc. which contacts the sample fluid. Many other possibilities exist and will be readily recognized by those skilled in this art without departing from the invention. For example, the kit may include a combination of detecting agent which can be useful for cell sorting the progenitor cells of the present disclosure, as discussed elsewhere in the present document.

The term “promoter” as used herein is defined as a DNA sequence recognized by the synthetic machinery of the cell, or introduced synthetic machinery, required to initiate the specific transcription of a polynucleotide sequence.

As used herein, the term “promoter/regulatory sequence” means a nucleic acid sequence which is required for expression of a gene product operably linked to the promoter/regulatory sequence. In some instances, this sequence may be the core promoter sequence and in other instances, this sequence may also include an enhancer sequence and other regulatory elements which are required for expression of the gene product. The promoter/regulatory sequence may, for example, be one which expresses the gene product in a tissue specific manner.

As used herein, the term “transcription factor” refers to a protein that controls the rate of transcription of genetic information from DNA to messenger RNA, by binding to a specific DNA sequence. In turn, this helps to regulate the expression of genes near that sequence.

By the term “specifically binds,” as used herein with respect to an antibody, is meant an antibody which recognizes a specific antigen, but does not substantially recognize or bind other molecules in a sample. For example, an antibody that specifically binds to an antigen from one species may also bind to that antigen from one or more species. But, such cross-species reactivity does not itself alter the classification of an antibody as specific. In another example, an antibody that specifically binds to an antigen may also bind to different allelic forms of the antigen. However, such cross reactivity does not itself alter the classification of an antibody as specific. In some instances, the terms “specific binding” or “specifically binding,” can be used in reference to the interaction of an antibody, a protein, or a peptide with a second chemical species, to mean that the interaction is dependent upon the presence of a particular structure (e.g., an antigenic determinant or epitope) on the chemical species; for example, an antibody recognizes and binds to a specific protein structure rather than to proteins generally. If an antibody is specific for epitope “A”, the presence of a molecule containing epitope A (or free, unlabeled A), in a reaction containing labeled “A” and the antibody, will reduce the amount of labeled A bound to the antibody.

As used herein, a “purified cell population” refers to a cell population which has been processed so as to separate the cell population from other cell populations with which it is normally associated in its naturally occurring state. The purified cell population can, thus, represent an enriched cell population in that the relative concentration of the cell population in a sample can be increased following such processing in comparison to its natural state. In one embodiment, the purified cell population can refer to a cell population which is enriched in a composition in a relative amount of at least 80%, or at least 90%, or at least 95% or 100% in comparison to its natural state. Such purified cell population may, thus, represent a cell preparation which can be further processed so as to obtain commercially viable preparations. For example, in one embodiment, the cell preparation can be prepared for transportation or storage in a serum-based solution containing necessary additives (e.g., DMSO), which can then be stored or transported in a frozen form. In doing so, the person of skill will readily understand that the cell preparation is in a composition that includes a suitable carrier, which composition is significantly different from the natural occurring separate elements. For example, the serum-based preparation may comprise human serum or fetal bovine serum, which is a structural form that is markedly different from the form of the naturally occurring elements of the preparation. The resulting preparation includes cells that are in dormant state, for example, that may have slowed-down or stopped intracellular metabolic reactions and/or that may have structural modifications to its cellular membranes. The resulting preparation includes cells that can, thus, be packaged or shipped while minimizing cell loss which would otherwise occur with the naturally occurring cells. A person skilled in the art would be able to determine a suitable preparation without departing from the present disclosure.

The composition described herein may include one or more pharmaceutically acceptable carrier. As used herein, the term “carrier” refers to any carrier, diluent or excipient that is compatible with the herein described NePs and can be given to a subject without adverse effects. Suitable acceptable carriers known in the art include, but are not limited to, water, saline, glucose, dextrose, buffered solutions, and the like. Such a carrier is advantageously non-toxic to the NePs and not harmful to the subject. It may also be biodegradable. The carrier may be a solid or liquid acceptable carrier. A suitable solid acceptable carrier is a non-toxic carrier. For instance, this solid acceptable carrier may be a common solid micronized injectable such as the component of a typical injectable composition for example, but without being limited to, kaolin, talc, calcium carbonate, chitosan, starch, lactose, and the like. A suitable liquid acceptable carrier may be, for example, water, saline, DMSO, culture medium such as DMEM, and the like. The person skilled in the art will be able to determine a suitable acceptable carrier for a specific application without departing from the present disclosure.

As used herein, the term “about” for example with respect to a value relating to a particular parameter (e.g. concentration, such as “about 100 mM”) relates to the variation, deviation or error (e.g. determined via statistical analysis) associated with a device or method used to measure the parameter. For example, in the case where the value of a parameter is based on a device or method which is capable of measuring the parameter with an error of ±10%, “about” would encompass the range from less than 10% of the value to more than 10% of the value.

The invention is further illustrated by the following non-limiting examples.

### EXAMPLES

The following examples describe some exemplary modes of making and practicing embodiments of the invention. It should be understood that these examples are for illustrative purposes only and are not meant to limit the scope of the compositions and methods described herein.

The following materials and methods were used to perform the practical examples described subsequently.

### Animal Husbandry and Ethics

C57BL/6J, B6 CD45.1 congenic mice, and NSG-SGM3 mice were purchased from The Jackson Laboratory. Mice were fed a standard rodent chow diet and were housed in microisolator cages in a pathogen-free facility. Mice were euthanized by CO₂ inhalation followed by cervical dislocation. All experiments followed approved guidelines of the La Jolla Institute for Allergy and Immunology Animal Care and Use Committee, and approval for use of rodents was obtained from the La Jolla Institute for Allergy and Immunology according to criteria outlined in the Guide for the Care and Use of Laboratory Animals from the National Institutes of Health.

Animals were randomly assigned to groups from available mice bred in our facility or ordered from distributor. Experiments in this study used male animals 6-10 weeks of age in good health. If animals were observed with non-experiment related health conditions (i.e. malocclusion, injuries from fighting, etc.), animals were removed from study groups.

### Human Bone Marrow Cells

Fresh BM samples of anonymous healthy adult donors were obtained from AllCells, Inc. (Alameda, Calif.). The cells were stained for either flow cytometry or FACS-sorting following protocols described in the Flow Cytometry and Cell Sorting section.

### Cell Culture

For tumor studies, B16F10 melanoma cells and 143B human osteosarcoma cells were obtained from ATCC. Cell lines were tested for being pathogen free. Cell lines were maintained in DMEM medium containing 10% heat-inactivated FBS, 2 mmol/L 1-glutamine, 1 mmol/L sodium pyruvate, 50 U/mL penicillin, 50 μg/mL streptomycin.

### Melanoma Patient Blood Collection

Melanoma patient (no previous radiation, no prior chemo treatment) blood collected in EDTA-tubes were provided by the Biospecimen Repository Core Facility (BRCF) from University of Kansas Cancer Center. Cells were stained for flow cytometry followed by the protocol described in the Flow Cytometry and Cell Sorting section.

### Human Peripheral Blood Collection

Heparinized blood from healthy volunteers was obtained after written informed consent under the guidelines of the Institutional Review Board of the La Jolla Institute for Allergy and Immunology and in accordance with US Dept. of Health and Human Services Policy for protection of Human Research Subjects (VD-057-0217). Cells were stained for flow cytometry followed by the protocol described in the Flow Cytometry and Cell Sorting section.

### Cell Suspension for Mass Cytometry and Flow Cytometry

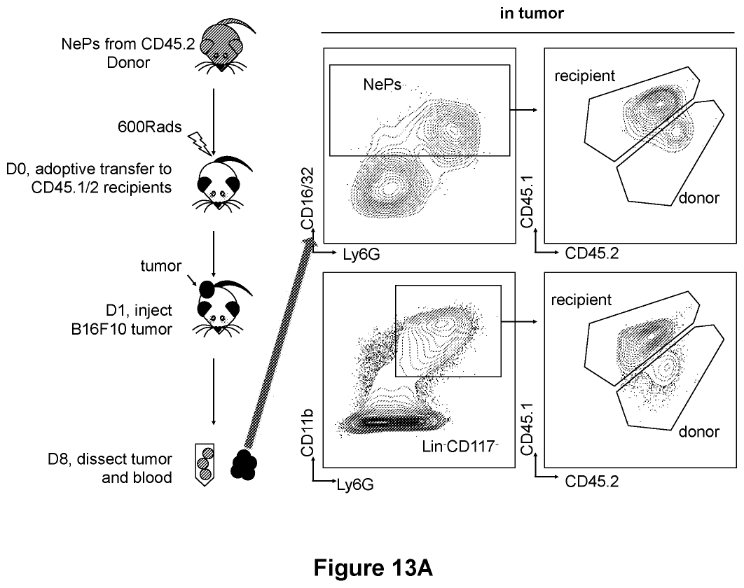

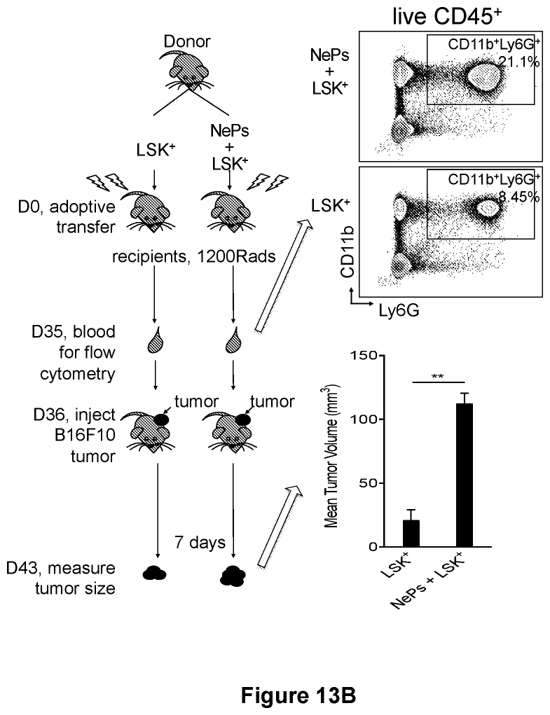

Bone marrow (BM) cells were harvested from femurs, and tibias of 6-10 week old mice. Bones were centrifuged for the collection of marrow. For the adoptive transfer experiments, donor BM cells were collected and stained under sterile conditions. Peripheral blood was obtained by cardiac puncture with an ethylenediaminetetraacetic acid (EDTA)-coated syringe. For FIG. 13A, a drop of blood was obtained from the saphenous vein of the adoptive transferred recipients. All samples were collected in ice cold DPBS (Dulbecco's phosphate buffered saline, Gibco) with 2 mM EDTA to prevent cation-dependent cell-cell adhesion. Prior to staining cells, cells were subject to a red blood cell lysis (RBC lysis buffer, eBiosciences) at room temperature (5 min×1 for BM cells, 10 min×2 for blood cells). Cells were washed and filtered through a 70 μm strainer. Cell suspensions were prepared by sieving and gentle pipetting to reach final concentration of 3×10⁶ cells per 100 μl buffer.

### Mass Cytometry Antibodies

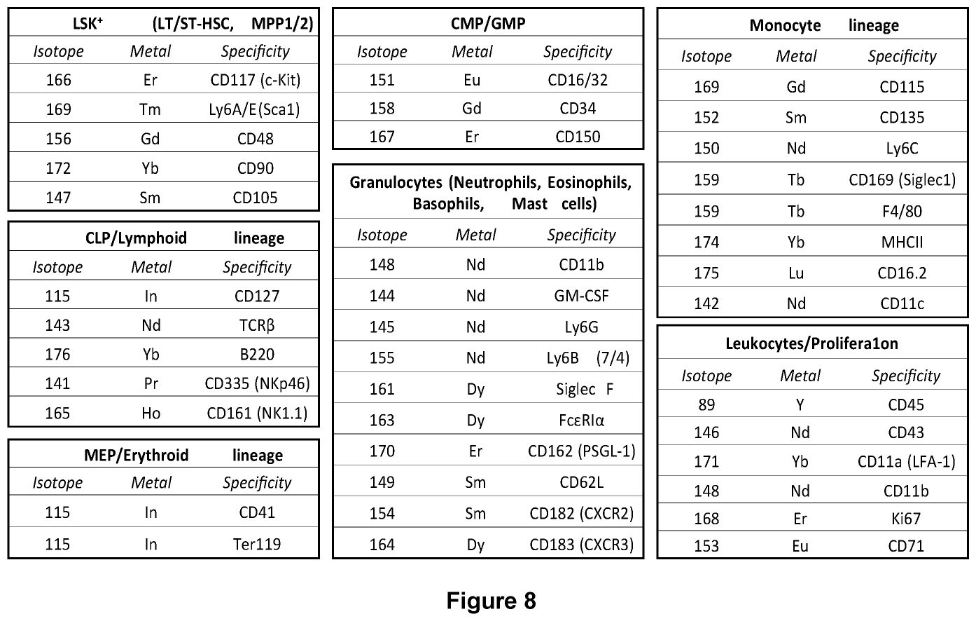

Metal-conjugated antibodies were purchased directly from Fluidigm™ for available targets. For all other targets, purified antibodies were purchased from the companies listed as provided in FIG. 8. Antibody conjugations were prepared using the Maxpar™ Antibody Labeling Kit according to the recommended protocol provided by Fluidigm. Maxpar-conjugated antibodies were stored in PBS-based antibody stabilization solution (Candor Biosciences) supplemented with 0.05% NaN₃ at 4° C. All antibodies were titrated before use.

### Mass Cytometry (CyTOF)

For viability staining, cells were washed in PBS and stained with Cisplatin (Fluidigm) to a final concentration of 5 μM. Prior to surface staining, anti-CD16/32 (151Eu) antibody was added to cell suspension in ice-cold staining buffer (PBS+2 mM EDTA+0.1% BSA+0.05% NaN₃) to stain and block the Fc receptors for 15 min. The surface antibody cocktail listed in FIG. 8 was then added into the cell suspension for 1 h. The cells were then washed and fixed with 2% paraformaldehyde overnight at 4° C. After fixation, cells were washed in staining buffer and permeabilized using Foxp3/Transcription Factor Staining Buffer (eBioscience) for intracellular staining according to the manufacturer's protocol. Following permeabilization, cells were washed twice with 1 ml 1× Perm Buffer (Saponin-based). The intracellular antibody cocktail listed in FIG. 8 were added into cell suspension for 1 h. For cell identification, cells were then washed in staining buffer and stained with DNA intercalator (Fluidigm) containing natural abundance Iridium (191Ir and 193Ir) prepared to a final concentration of 125 nM in 2% paraformaldehyde. Cells were washed in staining buffer, with subsequent washes in Milli-Q™ water (EMD Millipore) to remove buffer salts. Cells were resuspended in Milli-Q water with a 1:10 dilution of EQ™ Four Element Calibration beads (Fluidigm) and filtered through a 35 μm nylon mesh filter cap (Corning, Falcon). Samples were acquired on a Helios™, CyTOF® Mass Cytometer (Fluidigm) equipped with a Super Sampler (Victorian Airship & Scientific Apparatus) at an event rate of 500 events/second or less. Mass cytometry data files were normalized using the bead-based Normalizer (Finck et al, Cytometry A 83:48) and analyzed using Cytobank analysis software (the reader is referred to the Cytobank Internet website). The PhenoGraph clustering (Levine et al., 2015) and isomap dimensionality reduction were done using R package cytofkit (Chen et al., 2016). Hierarchical clustering was used to determine two meta-clusters based on the median of markers' expression from each PhenoGraph clusters.

### Flow Cytometry Antibodies

Antibodies for flow cytometry were purchased from commercial sources as follows: anti-CD3E (145-2C11; BD Biosciences); anti-CD19 (1D3; BD Biosciences); anti-CD161 (PK136; eBiosciences); anti-F4/80 (T45-2342; BD Biosciences); anti-CD11c (HL3; BD Biosciences); anti-CD45 (30-F11; BioLegend); anti-CD45.1 (A20; BioLegend); anti-CD45.2 (104; BioLegend); anti-CD117 (c-kit) (2B8; BioLegend); anti-Ly6A/E (Sca-1) (D7; BioLegend); anti-CD16/32 (FcγRIII/II (93; BioLegend); anti-CD11b (M1/70; BioLegend); anti-CD115 (M-CSFR) (AFS98; BioLegend); anti-Ly6G (1A8; BioLegend); anti-Ly6C (HK1.4; BioLegend); anti-Siglec F (E50-2446; BD Biosciences); anti-FcERIα (MAR-1; BioLegend); anti-Ki67 (SolA15; eBiosciences); anti-hCD45 (2D1; BioLegend); anti-hCD3E (HIT3a; BD Biosciences); anti-hCD7 (MT701; BD Biosciences); anti-hCD161 (HP-3G10; BioLegend); anti-hCD56 (B159; BD Biosciences); anti-hCD19 (HIB19; BD Biosciences); anti-hCD127 (A019D5; BioLegend); anti-hSiglec 8 (7C9; MACS Miltenyi Biotec); anti-hFcεRIα (AER-37; BioLegend); anti-hCD235a (GA-R2; BD Biosciences); anti-hCD41 (HIPS; BD Biosciences); anti-hCD169 (7-239; BD Biosciences); anti-hCD69 (10.1; BioLegend); anti-hCD11c (B-ly6; BD Biosciences); anti-hCD90 (5E10; BioLegend); anti-hCD86 (IT2.2; BioLegend); anti-hCD66B (G10F5; BioLegend); anti-hCD34 (581; BD Biosciences); anti-hCD117 (YB5.B8; BD Biosciences); anti-hCD38 (HB-7; BioLegend). Cell viability was determined with LIVE/DEAD™ Fixable Yellow (or Blue) Dead Cell Stain Kit (ThermoFisher).

### Flow Cytometry and Cell Sorting

All mouse FACS staining was performed in FACS buffer (DPBS+1% BSA+0.1% sodium azide+2 mM EDTA) on ice. All human FACS staining was performed in FACS buffer (DPBS+1% human serum+0.1% sodium azide+2 mM EDTA) on ice. Cells were filtered through sterile 70 μm cell strainers to obtain a single cell suspension (30,000 cells per μl for flow cytometry analysis, 0.5-2×10⁷ per ml for sorting). Prior to surface staining, anti-CD16/32 (FITC) antibody (for mouse) or human Fc receptors blocking reagent (MACS' Miltenyi Biotec) was added for 15 min to stain and block the Fc receptors. Surface staining was performed for 30 minutes in a final volume of 500 μl for FACS sorts and 100 ul for regular flow cytometry. Cells were washed twice in at least 200 μl FACS buffer before acquisition. Cells were sorted using a FACS Aria™ II and Aria-Fusion (BD biosciences) and conventional flow cytometry using an LSRII or a LSR Fortessa™ (BD Biosciences). All flow cytometry was performed on live cells. Calculations of percentages of CD45⁺ immune cells were based on live cells as determined by forward and side scatter and viability analysis. All analyses and sorts were repeated at least 3 times, and purity of sorted fractions was checked visually and by FACS reanalysis of the surface markers. Data were analysed using Cytobank (the reader is referred to the Cytobank Internet website) and FlowJo™ (version 10.1r5).

### Confocal Microscopy

Cells were FACS-sorted and resuspended in PBS. Following fixation in 4% methanol-free formaldehyde in PBS for 10 min at room temperature, cells were washed with PBS and resuspended in 5% normal donkey serum, 0.3% Triton™ X-100 in PBS for one hour. Cells were then incubated with a rabbit anti-Ki67 monoclonal antibody (clone SP6, Abcam, 1:150) or negative control (normal rabbit IgG) in 1% bovine serum albumin and 0.3% Triton X-100 in PBS overnight at 4° C. Cells were washed twice with PBS and incubated with anti-rabbit IgG (H+L) F(ab′)2 fragment conjugated to Alexa™ Fluor 647 (Cell Signalling, #4414, 1:500) and Hoechst (1:1000 of 10 mg/ml solution) for one hour at room temperature. After washing, cells were adhered to poly-L-lysine coated #1.5H coverslips and embedded in Prolong™ Gold (Thermo Fisher). Samples were imaged with a Zeiss LSM780 and Leica SP8 confocal microscopes using a 63×/1.40 NA oil-immersion objective. Images were processed with ZEN or Leica HyVolution™ software and 3D reconstructions of DNA were created in Imaris™ software. The mean and integrated fluorescence intensity (select the one you will show) of Ki67 within the nuclear regions were calculated in Image-Pro™ Premier. To reduce Z-stretching confocal images were deconvolved with Huygens Essential. Analysis of the surface area, volume and sphericity was performed in Imaris software.

### Cell Morphology

Cytospins from sorted populations were fixed on slides with methanol, stained with solutions of May-Grünwald (eosin methylene blue) and Giemsa (eosin methylene blue; Merck) and analyzed on a Nikon Eclipse 80i microscope (Nikon).

### Adoptive Transfer

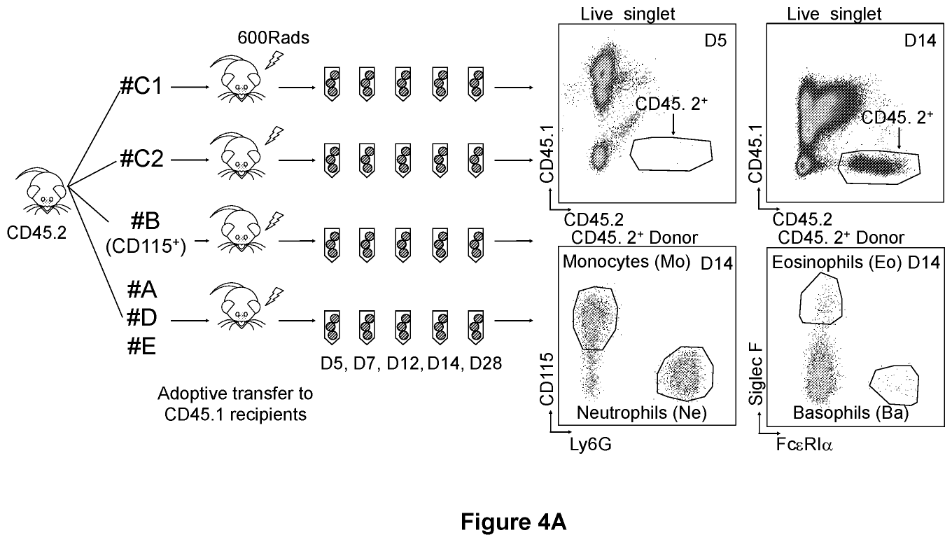

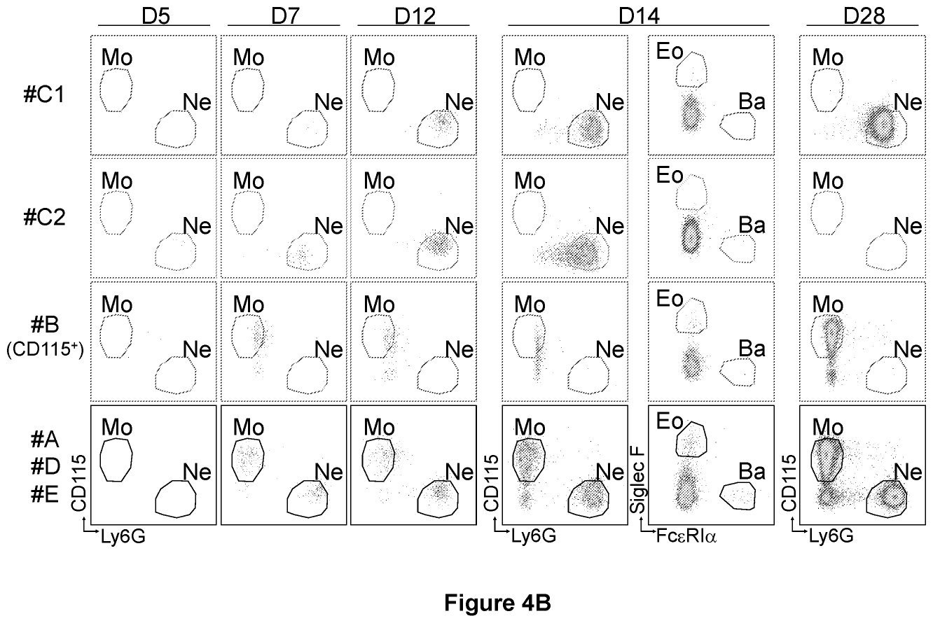

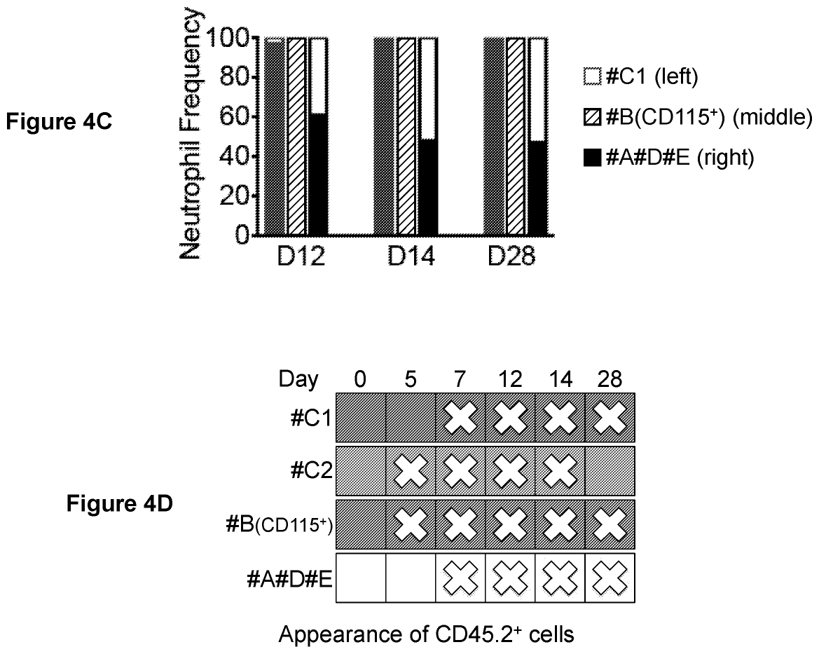

Recipient mice were housed in a barrier facility under pathogen-free conditions before and after adoptive transfer. NSG-SGM3 recipient mice were maintained in sterile conditions at all times. CD45.1 recipient mice were fed with autoclaved acidified water with antibiotics (trimethoprimsulfamethoxazole) for 3 days before the adoptive transfer. Sub-lethally irradiated recipient mice received 600 Rads. Donor BM cells were collected and FACS sorted as described in the flow cytometry section. Mouse and human progenitor cells were sorted directly into sterile FBS and kept chilled during sorting. Cells then were washed and resuspended in ice-cold DPBS for injection. 5×10⁴ donor progenitors in 200 μl DPBS were delivered into each recipient mouse for FIGS. 4-4D and FIGS. 5A-5B. 2.5×10⁴ donor progenitors in 200 μl DPBS were delivered into each recipient mouse for FIGS. 7A and 12A. All adoptive transfer experiments were achieved via tail vein injection. After the adoptive transfer, recipient mice were provided with autoclaved food and autoclaved acidified water with antibiotics.

### In Vitro Progenitor Differentiation Assay

Sorted progenitor cells (3×10⁴) were seeded into 6-well plates and cultured for 10 days with Methocult™ GF M3434 media (Stem Cell Technologies) according to the manufacturer's protocol. The numbers of wells containing proliferated colonies were counted for colony-forming assays.

### B16F10 Melanoma

For tumor injection, the hair around the tumor injection area of the 6-10 week old mice or adoptive transfer recipients was removed before injection. For FIG. 5A, 5×10⁵ B16F10 cells were washed and resuspended in 100 μl DPBS and then SubQ injected into the rear flank of the mouse, and the tumor-bearing mice were euthanized by CO₂ inhalation followed by cervical dislocation at Day 14 post-tumor injection. For FIG. 5B and FIG. 9A, 3×10⁵ B16F10 cells were washed and resuspended in 100 μl DPBS and then SubQ injected into the rear flank of the mouse, and the tumor size were measured with a digital caliper at Day 12 post-tumor injection. For FIG. 7C, 1×10⁶ 143B human osteosarcoma cells were washed and resuspended in 100 μl DPBS and then SubQ injected into the rear flank of the mouse, and the tumor size were measured with a digital caliper at Day 10 post-tumor injection. Tumor volume was calculated using the formula V (volume)=D×d²/2 (D is the largest measured tumor diameter and d is the smallest measured tumor diameter). Laboratory personnel were blinded to the identities of experimental groups during sample collection and analysis.

### Single-Cell RNA-Seq. 3′ End

Single cell RNA-Sequencing was performed using Chromium™ Single Cell 3′ v2 Reagent Kits (10× Genomics) following the manufacturer's protocol (Zheng et al., 2017). Briefly, after sort collection, cells were resuspended in PBS at concentration ranging between 400 to 600 cells per μ1. Between 5,000 to 10,000 cells were loaded for gel bead-in-emulsion generation and barcoding. To increase barcode diversity, samples were split in 2 technical replicates for all downstream steps: Reverse transcription, cDNA amplification, fragmentation and library preparation. Final libraries with size ranging between 200 to 1000 bp were size-selected using AMPure™ XP beads (Beckman Coulter). Quality and quantity of samples was controlled at multiple steps during the procedure by running small fraction (<5%) of sample on BioAnalyzer™ (high sensitivity DNA chip, Agilent). Libraries were sequenced on HiSeq2500 platform to obtain 26 (read1)×100 (read2) paired-end reads.

### Single Cell RNA-Seq Analysis

Using Cell Ranger v1.3.0 (10× genomics), reads were aligned on the mm10 reference genome for mouse and hg19 reference genome for human and unique molecular identifier gene expression profiles were generated for every single cell reaching standard sequencing quality threshold (default parameters). On average we obtained data for 2868 cells for mouse samples and 518 cells for human samples, and on average 46,477 reads per cell for mouse and 274,080 reads per cell for human. Only confidently mapped, non-PCR duplicates with valid barcodes and UMIs were used to generate a gene-barcode matrix for further analysis. Counts were normalized to get counts per million (CPM). Unbiased clustering of single cells was performed using Seurat (version 1.4) (R Development Core Team, 2016; Satija et al., 2015). Principal Component Analysis (PCA) was performed using a set of top variable genes (ranging between 647 to 2142 genes) and then dimensionality reduction was performed using t-SNE algorithm with top 10 to 18 PCAs. For FIG. 2A, tSNE 2D plots were obtained applying Seurat scRNA-Seq analysis R Package (using 12 first PCA, and 810 most variable genes with resolution parameter set at 0.03).

### Quantitative Real-Time PCR

RNA purity and quantity was measured with a Nanodrop™ spectrophotometer (Thermo Scientific). Approximately 100 ng RNA was used for synthesis of cDNA with an Iscript™ cDNA Synthesis Kit (Bio-Rad). Total cDNA was diluted 1:20 in H₂O, and a volume of 9 μl was used for each real-time condition with a MyIQ™ Single-Color Real-Time PCR Detection System (Bio-Rad) and TaqMan® Gene Expression Mastermix and TaqMan primers (Life Technologies). Data were analyzed and presented on the basis of the relative expression method. β-actin was used as ‘housekeeping’ gene for data normalization.

### Statistical Analysis

Data for all experiments were analyzed with Prism™ software (GraphPad). Unpaired t-tests and two-way analysis of variance were used for comparison of experimental groups. P values of *P<0.05, **P<0.01 were considered to indicate statistical significance. The data appeared to be normally distributed with similar standard deviation and error observed between and within experimental groups. No statistical methods were used to predetermine sample size. No animal or sample was excluded from the analysis.

### Example 1

In this example, the inventors demonstrate that the neutrophil progenitor cell population of the present disclosure can be extracted from a biological sample, in particular a mouse bone marrow (BM) sample.

FIG. 1A to 1C as a whole, show a gating strategy using mass cytometry defining a largest Cluster #C of the 5 subsets in Lin⁻ CD117⁺Ly6A/E⁻ cells from murine BM. BM cells isolated from C57BL/6J donors were stained with the antibody panel shown in FIG. 8.

Using mass cytometry, the inventors developed an antibody panel shown in FIG. 8 that measures 39 parameters simultaneously and used it to perform CyTOF mass cytometry on healthy mouse bone marrow. To solely focus on myeloid cell progenitors, the inventors analyzed the Lin⁻ CD117⁺ Ly6A/E⁻ fraction of LK cells by CyTOF using this panel. viSNE automated analysis was used to find 5 distinct clusters of cells, labeled as Clusters #A-E in FIG. 1A. Each of these clusters expresses distinctive biomarkers that uniquely define specific myeloid cell types. Siglec F (cluster #A) marks eosinophils, CD115 (cluster #B) marks monocytes, Ly6G (cluster #C) marks neutrophils, FcERIα (cluster #D) marks mast cells and basophils, and CD16/32 and CD34 (cluster #E) marks both CMP and GMP. The neutrophil-specific antigen, Ly6G, is observed in a continuum from negative to high expression in Cluster #C, showing the presence of neutrophil progenitors and immature neutrophils within this cluster (Kim et al., 2017; Satake et al., 2012; Sturge et al., 2015; Yáñez et al., 2015).

To identify neutrophil progenitors, the inventors focused efforts on further analysis of Cluster #C. Using Phenograph, a second unbiased clustering algorithm (Chen et al., 2016; Levine et al., 2015), it was found that Cluster #C consists of two major populations that display a continuum of Ly6G, Ly6C, and Ly6B expression (FIG. 1B). These Ly6 proteins are highly expressed in neutrophils and precursors (Kim et al., 2017; Lee et al., 2013). A conventional flow cytometry gating strategy shown in FIG. 1C was developed to isolate with purity Cluster #C cells (Lin⁻ CD117⁺Ly6A/E⁻ Siglec F⁻ FcERIα⁻ CD16/32⁺Ly6B⁺ CD162^{lo} CD48^{lo} Ly6C^{lo} CD115⁻) from bone marrow. This cell population, when backgated onto a viSNE map fell exclusively into Cluster #C (FIG. 1C).

### Example 2

In this example, the inventors used scRNA Seq analysis of Cluster #C to reveal two major subpopulations (#C1 and #C2).

The inventors further investigated Cluster #C by sorting Cluster #C from mouse BM for scRNA-Seq analysis using the gating strategy in FIG. 1C. The Seurat algorithm was used to analyze scRNA-Seq data (Rizzo, 2016; Satija et al., 2015). Automated clustering of Cluster #C showed the presence of two primary subpopulations within Cluster #C, #C1 and #C2 (FIG. 2A). These two subpopulations show differential expression of key genes that are important for neutrophil as well as myeloid cell development. Gfi1 is critical for neutrophil development (Horman et al., 2009). Peri and Ets1 are associated with Gfi1 expression by single-cell analysis of Gfi1−/− GMP, demonstrating collaboration of these genes in controlling granulocyte development (Olsson et al., 2016). Clusters #C1 and #C2 clusters globally express Gfi1 and Cebpa with a higher mean value in #C1. Disruption of C/EBPα expression and function absolutely blocks granulopoiesis (Radomska et al., 1998; Zhang et al., 1997) and greatly impairs neutrophil differentiation (Avellino et al., 2016). Compared to #C1, the #C2 cluster showed reduced expression of known myeloid transcription factors including Tfec and Myb (Friedman, 2007; Olsson et al., 2016; Zhu et al., 2016). #C1 and #C2 also show differential Ly6g expression (FIG. 2A, bottom), which confirms the mass cytometry data shown in FIG. 1B.

Next, a flow cytometry panel shown in FIG. 2B was generated to isolate #C1 and #C2 as well as other Lin⁻ CD117⁺Ly6A/E⁻ cell fractions. The purity of the gated populations from this manual gating strategy was validated by backgating them to the viSNE map. Cluster #C1 is Lin⁻ CD117⁺Ly6A/E⁻ Siglec F⁻ FcERIα⁻ CD16/32⁺Ly6B⁺CD11a⁺ (LFA1α⁺) CD162^{lo} CD48^{lo} Ly6C^{lo} CD115⁻Ly6G⁻ and cluster #C2 is Lin⁻CD117⁺Ly6A/E⁻ Siglec F⁻ FcERIα⁻ CD16/32⁺Ly6B⁺CD11a⁺ (LFA1α⁺) Ly6G⁺.

### Example 3

In this example, the inventors show that Cluster #C1 cells are unipotent neutrophil progenitors in vitro.

Comparison of #C1 and #C2 showed a gradient of Ly6G expression from negative in #C1 to intermediate in #C2 to high in mature BM Neuts (FIG. 3A). Reconstruction in 3-D of the nuclear architecture of #C1 and #C2 cells demonstrates more stem-cell like morphology than that of mature BM Neuts and Blood Neuts (FIG. 3B). #C1 has more stem cell-like nuclear morphology and higher Ki67 expression and nuclear integration (FIG. 3C) than does #C2, BM Neuts and Blood Neuts, showing an early stage of development for #C1.

The selective neutrophil potency of #C1 cells was first tested by examining in vitro methylcellulose colony-forming unit formation (FIG. 3D). All donor cell fractions were FACS sorted using the gating strategy described in FIG. 2B. CD115⁺CD117⁺ cells are monocyte progenitors and are located within Cluster #B therefore the CD115⁺ portion of Cluster #B was sorted as monocyte progenitors (FIG. 9B). Clusters #A, D, E were collected together as a control group. As shown in FIG. 3D, #C1 single cells generate colony-forming unit-granulocyte (CFU-G) in methylcellulose-based medium with 100% purity, but not colony-forming unit-macrophage (CFU-M) or colony-forming unit-granulocyte, macrophage (CFU-GM). Cluster #B (CD115⁺) cells were able to generate CFU-M only, as expected. The #A #D #E control group generated all three types of colonies.

### Example 4

In this example, the inventors describe a functional analysis of the progenitor cell population of the present disclosure, showing the Cluster #C1 is the early-stage committed unipotent neutrophil progenitor (NeP) in vivo.

The function of #C1 in generating neutrophils in vivo was analyzed using adoptive transfer approaches. The experimental scheme is shown in FIG. 4A. The cell populations described in FIG. 3D were FACS sorted from the same donors. #C2 was also sorted for this experiment to evaluate its neutrophil potency. These 4 cell groups were adoptively transferred into 4 groups of sub-lethally irradiated CD45.1 recipient mice. Blood from each group was examined at days 5, 7, 12, 14 and 28 by flow cytometry for appearance of donor-derived progeny. The flow cytometry gating for these donor cell progeny is shown in representative plots of the #A #D #E recipient group in FIG. 4A right panel. Donor cells (CD45.2⁺) appeared in blood as early as day 5 and peaked at day 14. Donor cells were analyzed for expression of key markers for myeloid progenies: monocytes (Mo, CD115⁺), neutrophils (Ne, Ly6G⁺), eosinophils (Eo, Siglec F⁺), or basophils (Ba, FcERIα⁺).

Donor-derived neutrophils appeared in recipient blood at Day 5 and Day 7 post-adoptive transfer in the groups reconstituted with #C1 and #C2, showing neutrophil potency in both populations and slower kinetics of the #C1 cells in producing neutrophils (FIG. 4B). Neutrophil progenies from these progenitors comprise nearly 100% of CD45.2⁺ donor cell-derived leukocytes in the #C1 recipients (FIGS. 4B and 4C). In the control groups, #B (CD115⁺) only produced monocytes and did not produce neutrophils and #A #D #E produced both neutrophils and monocytes (FIGS. 4B and 4C). This result illustrates the unipotency of #C1 and #C2 progenitors to restrictedly generate solely neutrophils.

Neutrophil production peaks at day 14 in #C2 recipients but at day 28, neutrophils vanished from the #C2 recipients, showing limited developmental potency of #C2 (FIGS. 4B and 4D). However, in #C1 recipients, neutrophil production continued to day 28, showing that the #C1 progenitors have longer-term potency. This long-term potency of #C1 is comparable to the #A #D #E fractions of Lin⁻ CD117⁺ Ly6A/E⁻ cells which contains CMP, again confirming that #C1 is the early-stage committed neutrophil progenitor.

Taken together, by using high dimensional mass cytometry and scRNA-Seq the inventors have discovered an early-stage committed neutrophil progenitor (#C1, termed NeP) in mouse bone marrow. This progenitor can be identified as Lin⁻ CD117⁺Ly6A/E⁻ Siglec F⁻ FcERIα⁻ CD16/32⁺ Ly6B+CD11a⁺ CD162^{lo} CD48^{lo} Ly6C¹⁰ CD115⁻Ly6G⁻.

### Example 5

In this example, the inventors further describe a functional analysis of the progenitor cell population of the present disclosure in the context of tumor growth.

Granulopoiesis is often associated with cancer (3). The inventors examined whether #C1 NeP progenitor cells were increased in the bone marrow and periphery of mice using a melanoma tumor model. B16F10 tumor cells SubQ were injected into the rear flank of wild-type C57BL/6J mice (Tumor). Age-matched, gender-matched wild-type mice received D-PBS to serve as healthy controls (Healthy). At 14 days post-injection, tissues were harvested for flow cytometry analysis. The inventors found an expansion of #C1 NeP progenitor cells, but not #E or #B (CD115⁺) cells, in the bone marrow of tumor-bearing mice (FIG. 5A), indicating that in the setting of cancer, this expansion is selective for NeP. Interestingly, the inventors detected minimal numbers of Cluster #C cells (less than 0.02% of all CD45⁺ cells in the periphery) of healthy mice, whereas #C cells are increased 10-fold in periphery of tumor-bearing mice (FIG. 9A). Without being bound to a particular theory, it is believed that there is increased production and egress of these neutrophil progenitors from bone marrow to periphery in response to the tumor microenvironment.

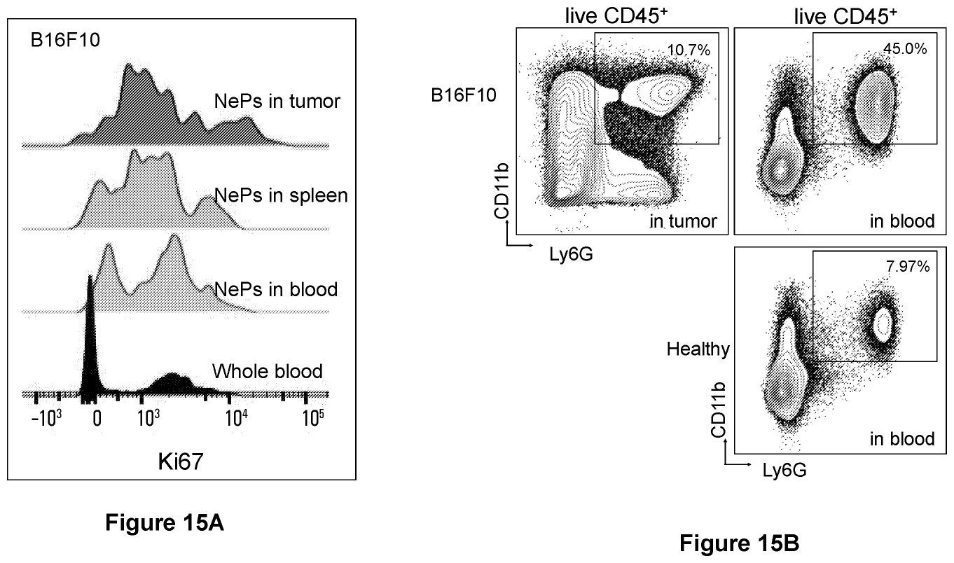

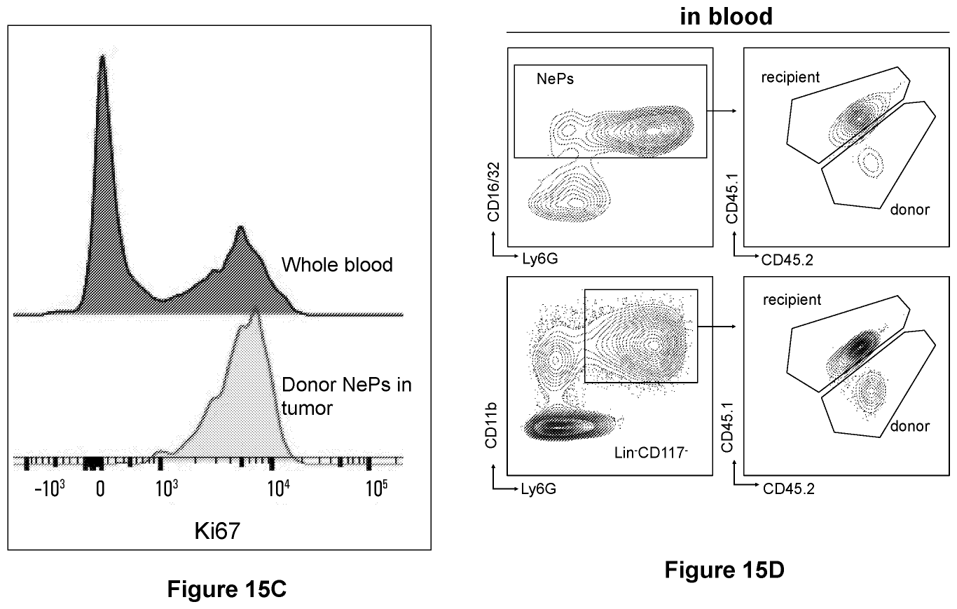

To test whether NePs can contribute to tumor growth, #C1 NeP cells, #B (CD115⁺) cells, and #E cells were sorted from CD45.2 wild-type donor mice and adoptively transferred into irradiated CD45.1 recipient healthy mice. At day 1 after donor cell transfer, recipient mice were injected SubQ with B16F10 tumor cells into the rear flank. Tumor size was measured at day 12 after injection (FIG. 5B, left). As shown in FIG. 5B, right, mice receiving #C1 NeP cells showed increased tumor growth compared to #B (CD115⁺) cells or #E cells. This data illustrates that #C1 NeP progenitors respond to melanoma tumor cues and have tumor-promoting functions. The inventors found that NePs in blood, spleen, and tumor of tumor-bearing mice were proliferative, as measured by Ki67 staining (FIG. 15A). Finally, it was observed that the tumor-bearing mice had CD11b+Ly6G+ cells in tumor (FIG. 15B, left) and increased in blood (FIG. 15B, right).

In further studies to confirm that NePs can be recruited directly to the tumor, NePs were sorted from CD45.2 wild-type donor mice and adoptively transferred into irradiated CD45.1/2 recipient healthy mice. At day 1 after donor NeP transfer, recipient mice were injected SubQ with B16F10 tumor cells into the rear flank. At day 8, the blood and early tumor were harvested for analysis (FIG. 13A, top panel). It was observed that donor-derived CD45.2 NePs appeared in the tumor (FIG. 13A, right panel top), and were proliferative (FIG. 15C). The donor-derived NePs also appeared in the blood (FIG. 15D top). Finally, the inventors found that these NePs also gave rise to CD11b+Ly6G+ cells, both in the tumor (FIG. 13A, right panel bottom) and in the blood (FIG. 15D bottom). These data indicate that 1) NePs are expanded in response to tumor, and can directly migrate to seed the blood, spleen, and tumor tissues, and 2) within the tumor environment, NePs produce progeny with surface markers similar to those that currently define MDSC.

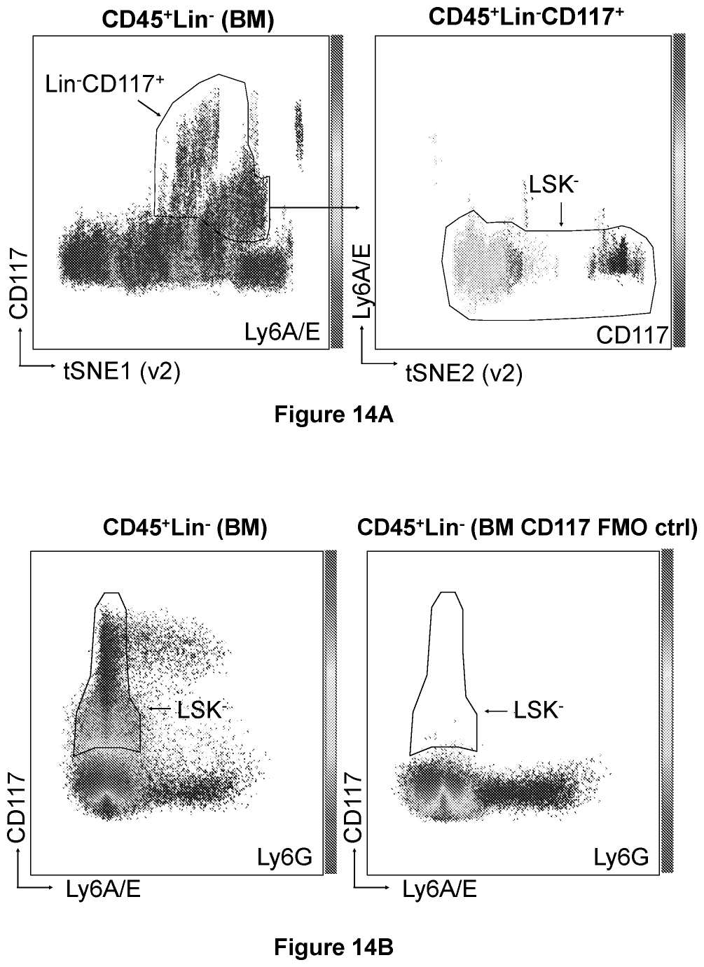

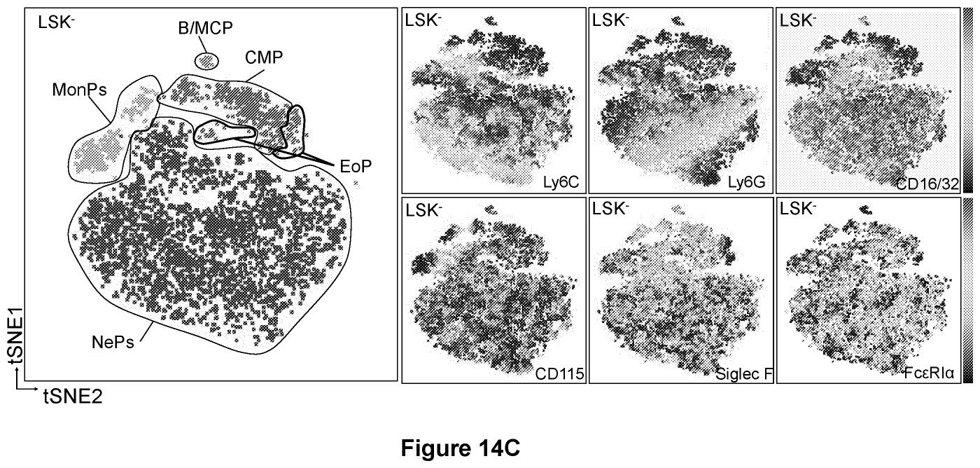

To directly investigate the role of NePs in driving tumor progression, the inventors performed a series of extended adoptive transfers in a cancer model in vivo (FIG. 13B). NePs, and LSK+ HSPCs were sorted from donor mice. NePs were co-transferred with LSK+ HSPCs into lethally irradiated recipient mice. LSK+ HSPCs were co-transferred in this model for full blood reconstitution to maintain healthy recovery of the recipients from irradiation. Mice that received LSK+ HSPCs served as a control group. At 35 days after adoptive transfer, blood was collected from each recipient to analyze CD45+ myeloid cell populations (FIG. 13B, left panel). In the NePs+LSK+ recipients, the CD11b+Ly6G+ population was over two-fold greater than the LSK+ only recipients (FIG. 13B, right panel top). B16F10 melanoma was then introduced via SubQ injection to these two groups of recipients and tumor sizes were measured 7 days later (FIG. 13B, left panel). Tumor volumes in the NePs+LSK+ recipients were about 5-fold larger than in the LSK+ only recipients (FIG. 13B, right panel bottom), demonstrating positive correlations linking NePs with neutrophil-favored myelopoiesis, and tumor growth. Lin⁻CD117⁺Ly6A/E− cells were gated carefully with the same methods used for mass cytometry data (FIGS. 13A, 13B and 14A). In addition, the CD117+ gate was distinguished from the CD117− gate by comparing it to a CD117 FMO stained BM sample specifically for flow cytometry data (FIG. 14B). The successful isolation of LSK− HSPCs with flow cytometry is confirmed with viSNE automated mapping which resulted in the same 5 cell subsets as the mass cytometry data (FIG. 14C).

### Example 6

In this example, the inventors show the discovery of a heterogeneous hCD66b⁺hCD117⁺hCD38⁺hCD34^{+/-} progenitor-like cell fraction in human bone marrow.

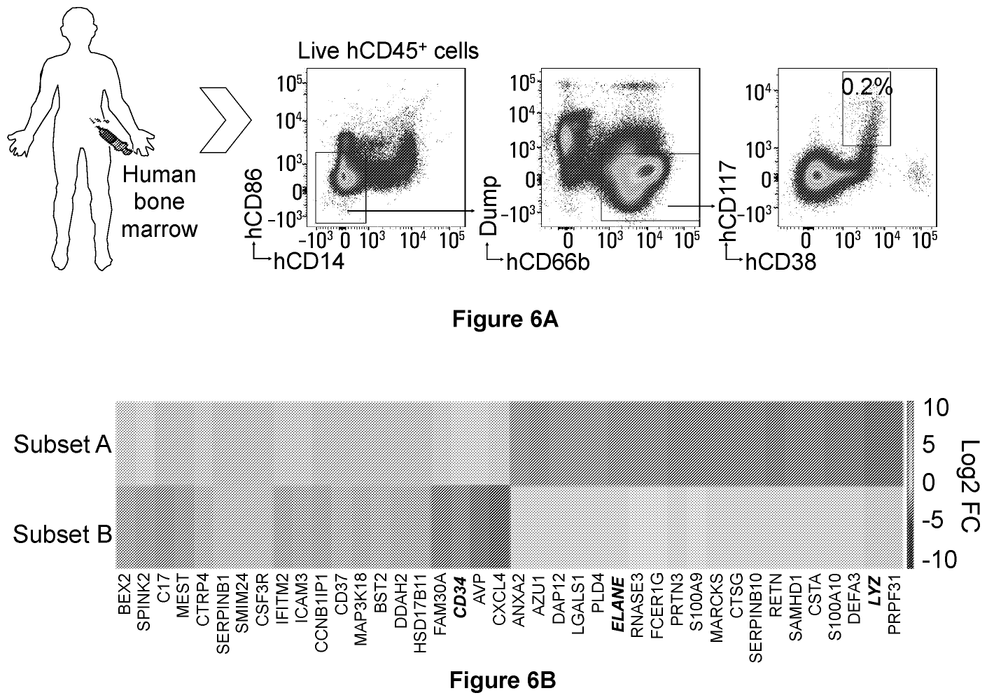

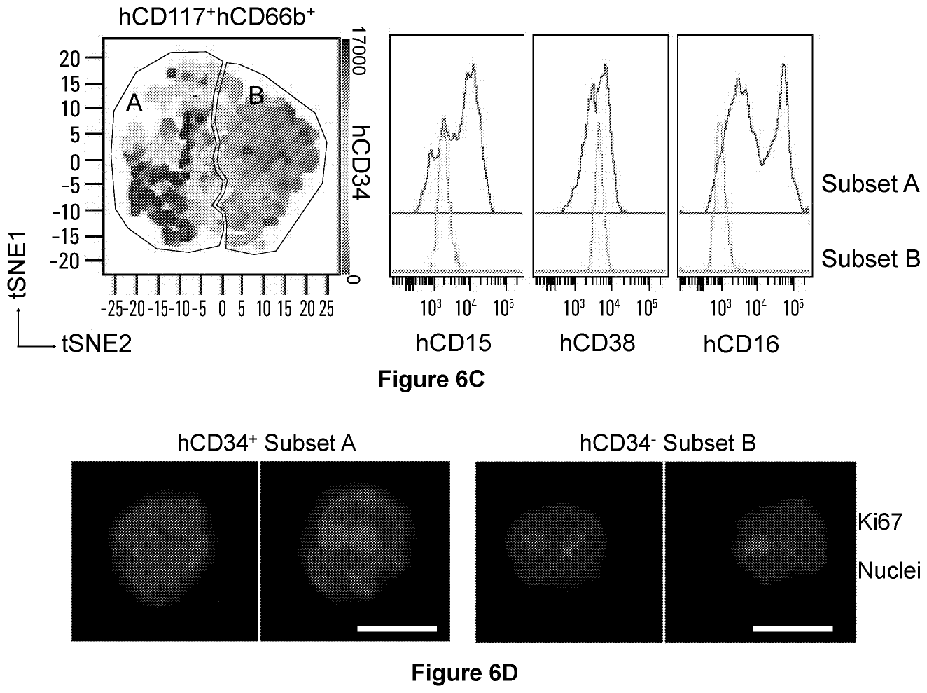

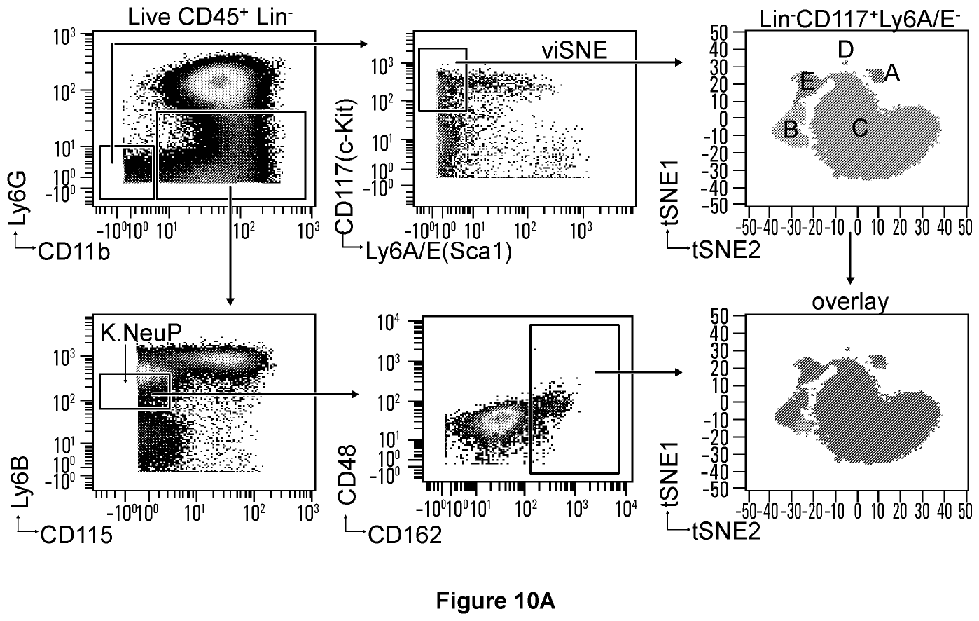

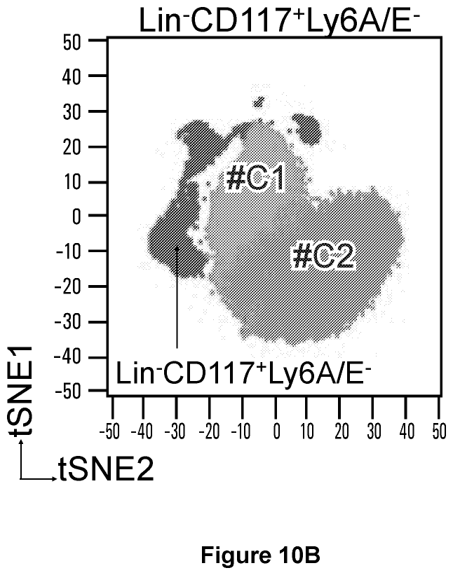

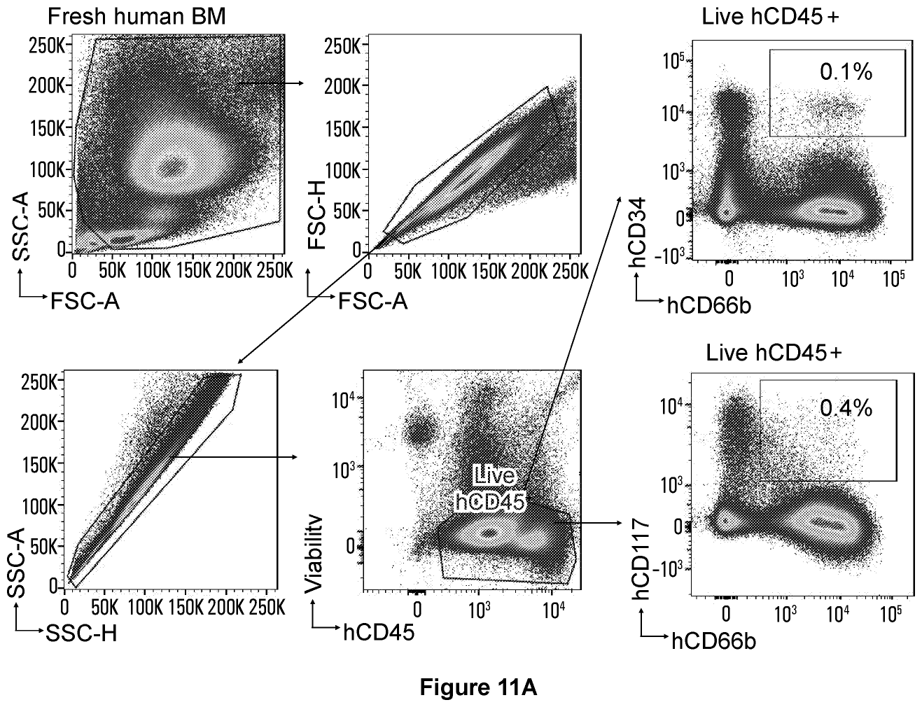

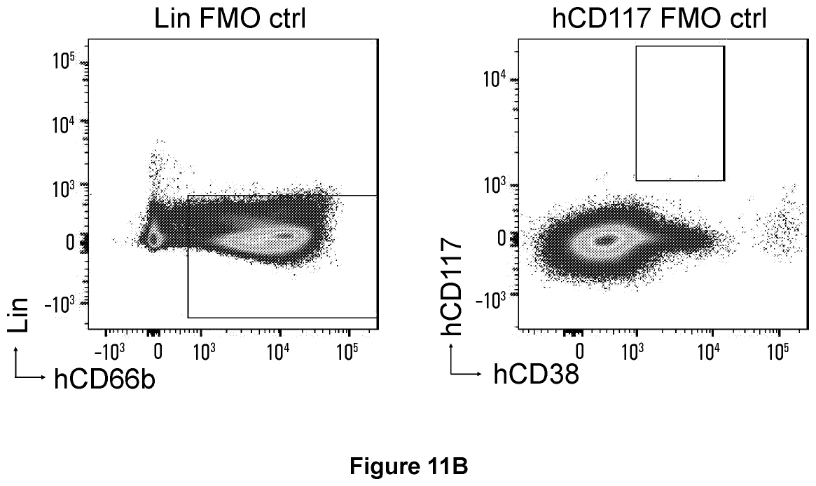

The inventors next analyzed healthy human bone marrow. Human CMP and GMP express hCD34, hCD38, and hCD117 and mirror the murine CMP/GMP paradigm in myeloid cell production (Doulatov et al., 2010; Edvardsson et al., 2006; Manz et al., 2002). CD66b is considered a marker of mature myeloid cells and, as such, is often excluded from flow cytometry panels geared towards hematopoietic progenitors. However, as this is an important marker for neutrophil identification, this marker was retained in the search for the early neutrophil progenitor in human bone marrow. Indeed, the inventors discovered that human bone marrow contains a heterogenous hCD66b⁺ population that expresses either CD34⁺ or CD117⁺ (FIG. 10A), demonstrating the presence of hCD66b⁺ stem cell progenitors within human bone marrow. A flow cytometry panel was developed to fully investigate these hCD66b⁺ progenitor populations (FIG. 6A), and validated with FMO controls (FIGS. 6A and 10B). Using this strict flow cytometry gating strategy, the inventors further identified a hCD66b⁺hCD117⁺ population of cells that expresses hCD38⁺ residing within this population that occupies about 0.2% of hCD45⁺ cells in human BM (FIG. 6A). ScRNA-Seq analysis of this hCD66b⁺hCD117⁺ human neutrophil progenitor population revealed two major subsets which showed either positive (Subset A) or negative (Subset B) expression of CD34 (FIG. 6B). Lower CD34 gene expression in Subset B is associated with increased expression of neutrophil-specific genes such as ELANE and LYZ (FIG. 6B). ViSNE analysis of these hCD66b⁺hCD117⁺hCD38⁺ cells also showed two major populations, one with high expression of hCD34 (fraction A in FIG. 6C) and one that is negative for hCD34 (fraction B in FIG. 6C). The two subsets express different levels of other markers including hCD15 and hCD16 (FIG. 6C). Both subsets appeared positive for Ki67 localization in the nuclei, showing active proliferation, with a slightly higher (about 1.3 fold) Ki67 mean fluorescence intensity value in hCD34⁺ Subset A compared to hCD34⁻ Subset B (FIG. 6D).

### Example 7

In this example, the inventors show both hCD66b⁺hCD117⁺hCD38⁺ subsets produce only neutrophils in NSG-SGM3 (NSG-M3) mice.

The inventors examined the neutrophil potency of these human neutrophil progenitor candidates (hCD34⁺ Subset A and hCD34⁻ Subset B) in vivo by performing adoptive transfers of each subset into NSG-SGM3 (NSG-M3) mice. The triple transgenic NSG-M3 mice are immunodeficient NOD scid gamma (NSG™) mice that express the human cytokines Interleukin 3 (IL-3), granulocyte/macrophage-stimulating factor (GM-CSF) and SCF, also known as KITLG. This mouse model supports stable engraftment of human myeloid lineages. The two subsets were isolated from fresh human bone marrow by FACS using the sorting panel in FIG. 6A and transferred into two groups of NSG-M3 mice, respectively. Peripheral blood of each NSG-M3 recipient mouse was collected at day 5, 7, 14 and 28 for flow cytometry analysis (FIG. 7A). Recipient blood was analyzed for monocyte (Mo), neutrophils (Ne), eosinophils (Eo), and lymphocytes (Ly) including T cells, B cells and NK cells with the flow cytometry panel shown in FIG. 12A. After adoptive transfer, hCD66b expression was detected in both hCD34⁺ Subset A and hCD34⁻ Subset B recipients and no other markers were positive (FIG. 7B and FIG. 12B), illustrating that both subsets are unipotent progenitors that produce only neutrophils. Repopulation of the neutrophil pool by either progenitor subset A or B occurred quickly after the adoptive transfer (day 5) and lasted to day 28 (FIG. 7B), indicating relatively long-term neutrophil unipotency of both progenitor subsets. These data demonstrate the hCD66b⁺hCD117⁺hCD38⁺hCD34^{+/-} fraction in human BM cells contains the unipotent human neutrophil progenitor (termed here as hNeP).

### Example 8

In this example, the inventors show hNeP increase in melanoma patient blood and promote early osteosarcoma tumor growth in humanized NSG-M3 mice.

The inventors examined whether hNeP played a role in tumorigenesis by examining osteosarcoma growth in NSG-M3 mice. Osteosarcoma is the most common type of cancer and is an important solid tumor target for immunotherapy (Anderson, 2017). Shown in FIG. 7C, left, both hCD34⁺ Subset A and hCD34⁻ Subset B were isolated from human BM and adoptively transferred into NSG-M3 recipient mice. Two different control groups were used in this experiment: one control group received only PBS for adoptive transfer, the other group received human cMoP as a source of human monocyte progenitors. Human cMoP were sorted from the same human BM donor using the panel described previously (Kawamura et al., 2017). One day one after adoptive transfer of progenitors, 1×10⁶ human osteosarcoma cells were injected SubQ to the rear flank of mice in all 4 recipient groups. The tumor size was measured 10 days after injection. As shown in FIG. 7C, right, mice receiving either hCD34⁺ Subset A or hCD34⁻ Subset B cells showed an increase in tumor growth compared to recipient mice receiving cMoP or PBS as a control. This data is concomitant with the mouse data shown in FIG. 5B, showing that hNeP, the counterpart of mouse NeP, also are pro-tumoral and mediate solid tumor growth.

Finally, blood from human subjects with melanoma was analyzed for the presence of hNeP. Blood specimens collected from patients prior to treatment who were diagnosed with melanoma were used. Flow cytometry analysis of healthy donor blood as well as melanoma patient blood using the panel in FIG. 6A revealed the presence of hCD66b⁺hCD117⁺ cells (about 1% of circulating hCD45⁺ cells) in blood of healthy donors (FIG. 7D). The frequency of these hNeP was significantly elevated in blood of melanoma patients, with frequencies of about 6% in circulating hCD45⁺ cells (FIG. 7C). This 5-6 fold increase of hNeP cells in human melanoma patient blood is consistent with what is observed in the mouse melanoma model (FIGS. 9A-9B), showing that the hNeP can serve as a biomarker candidate for early cancer detection.

Other examples of implementations will become apparent to the reader in view of the teachings of the present description and as such, will not be further described here.

Note that titles or subtitles may be used throughout the present disclosure for convenience of a reader, but in no way should these limit the scope of the invention. Moreover, certain theories may be proposed and disclosed herein; however, in no way they, whether they are right or wrong, should limit the scope of the invention so long as the invention is practiced according to the present disclosure without regard for any particular theory or scheme of action.

All references cited throughout the specification are hereby incorporated by reference in their entirety for all purposes.

It will be understood by those of skill in the art that throughout the present specification, the term “a” used before a term encompasses embodiments containing one or more to what the term refers. It will also be understood by those of skill in the art that throughout the present specification, the term “comprising”, which is synonymous with “including,” “containing,” or “characterized by,” is inclusive or open-ended and does not exclude additional, un-recited elements or method steps.

Unless otherwise defined, all technical and scientific terms used herein have the same meaning as commonly understood by one of ordinary skill in the art to which this invention pertains. In the case of conflict, the present document, including definitions will control.

As used in the present disclosure, the terms “around”, “about” or “approximately” shall generally mean within the error margin generally accepted in the art. Hence, numerical quantities given herein generally include such error margin such that the terms “around”, “about” or “approximately” can be inferred if not expressly stated.

With respect to ranges of values, the invention encompasses the upper and lower limits and each intervening value between the upper and lower limits of the range to at least a tenth of the upper and lower limit's unit, unless the context clearly indicates otherwise. Further, the invention encompasses any other stated intervening values.

Although various embodiments of the disclosure have been described and illustrated, it will be apparent to those skilled in the art in light of the present description that numerous modifications and variations can be made. The scope of the invention is defined more particularly in the appended claims.

### REFERENCES

1. C. Summers et al., Neutrophil kinetics in health and disease. Trends in immunology 31, 318 (August, 2010).
2. J. Huang, Y. Xiao, A. Xu, Z. Zhou, Neutrophils in type 1 diabetes. Journal of diabetes investigation 7, 652 (September, 2016).
3. C. Hagerling, Z. Werb, Neutrophils: Critical components in experimental animal models of cancer. Seminars in immunology 28, 197 (April, 2016).
4. K. Akashi, D. Traver, T. Miyamoto, I. L. Weissman, A clonogenic common myeloid progenitor that gives rise to all myeloid lineages. Nature 404, 193 (Mar. 9, 2000).
5. H. Iwasaki, K. Akashi, Myeloid lineage commitment from the hematopoietic stem cell. Immunity 26, 726 (June, 2007).
6. M. G. Manz, T. Miyamoto, K. Akashi, I. L. Weissman, Prospective isolation of human clonogenic common myeloid progenitors. Proceedings of the National Academy of Sciences of the United States of America 99, 11872 (Sep. 3, 2002).
7. V. Cortez-Retamozo et al., Origins of tumor-associated macrophages and neutrophils. Proceedings of the National Academy of Sciences of the United States of America 109, 2491 (Feb. 14, 2012).
8. A. J. Casbon et al., Invasive breast cancer reprograms early myeloid differentiation in the bone marrow to generate immunosuppressive neutrophils. Proceedings of the National Academy of Sciences of the United States of America 112, E566 (Feb. 10, 2015).
9. A. D. Amir el et al., viSNE enables visualization of high dimensional single-cell data and reveals phenotypic heterogeneity of leukemia. Nature biotechnology 31, 545 (June, 2013).
10. H. Iwasaki et al., Identification of eosinophil lineage-committed progenitors in the murine bone marrow. The Journal of experimental medicine 201, 1891 (Jun. 20, 2005).
11. J. Q. Zhang, B. Biedermann, L. Nitschke, P. R. Crocker, The murine inhibitory receptor mSiglec-E is expressed broadly on cells of the innate immune system whereas mSiglec-F is restricted to eosinophils. European journal of immunology 34, 1175 (April, 2004).
12. Y. Arinobu et al., Developmental checkpoints of the basophil/mast cell lineages in adult murine hematopoiesis. Proceedings of the National Academy of Sciences of the United States of America 102, 18105 (Dec. 13, 2005).
13. X. Qi et al., Antagonistic regulation by the transcription factors C/EBPalpha and MITF specifies basophil and mast cell fates. Immunity 39, 97 (Jul. 25, 2013).
14. D. K. Fogg et al., A clonogenic bone marrow progenitor specific for macrophages and dendritic cells. Science 311, 83 (Jan. 6, 2006).
15. K. Liu et al., In vivo analysis of dendritic cell development and homeostasis. Science 324, 392 (Apr. 17, 2009).
16. J. Hettinger et al., Origin of monocytes and macrophages in a committed progenitor. Nature immunology 14, 821 (August, 2013).
17. T. Satoh et al., Identification of an atypical monocyte and committed progenitor involved in fibrosis. Nature 541, 96 (Jan. 5, 2017).
18. S. Satake et al., C/EBPbeta is involved in the amplification of early granulocyte precursors during candidemia-induced “emergency” granulopoiesis. Journal of immunology 189, 4546 (Nov. 1, 2012).
19. C. R. Sturge, E. Burger, M. Raetz, L. V. Hooper, F. Yarovinsky, Cutting Edge: Developmental Regulation of IFN-gamma Production by Mouse Neutrophil Precursor Cells. Journal of immunology 195, 36 (Jul. 1, 2015).
20. A. Yanez, M. Y. Ng, N. Hassanzadeh-Kiabi, H. S. Goodridge, IRF8 acts in lineage-committed rather than oligopotent progenitors to control neutrophil vs monocyte production. Blood 125, 1452 (Feb. 26, 2015).
21. K. Fiedler, C. Brunner, The role of transcription factors in the guidance of granulopoiesis. American journal of blood research 2, 57 (2012).
22. B. Becher et al., High-dimensional analysis of the murine myeloid cell system. Nature immunology 15, 1181 (December, 2014).
23. N. Samusik, Z. Good, M. H. Spitzer, K. L. Davis, G. P. Nolan, Automated mapping of phenotype space with single-cell data. Nature methods 13, 493 (June, 2016).
24. S. C. Bendall et al., Single-cell mass cytometry of differential immune and drug responses across a human hematopoietic continuum. Science 332, 687 (May 6, 2011).
25. I. L. Weissman, D. J. Anderson, F. Gage, Stem and progenitor cells: origins, phenotypes, lineage commitments, and transdifferentiations. Annual review of cell and developmental biology 17, 387 (2001).
26. Y. P. Zhu, G. D. Thomas, C. C. Hedrick, 2014 Jeffrey M. Hoeg Award Lecture: Transcriptional Control of Monocyte Development. Arteriosclerosis, thrombosis, and vascular biology 36, 1722 (September, 2016).
27. S. R. Horman et al., Gfi1 integrates progenitor versus granulocytic transcriptional programming. Blood 113, 5466 (May 28, 2009).
28. R. Drissen et al., Distinct myeloid progenitor-differentiation pathways identified through single-cell RNA sequencing. Nature immunology 17, 666 (June, 2016).
29. Anderson, P. M. (2017). Immune Therapy for Sarcomas. Adv. Exp. Med. Biol. 995, 127-140.
30. Avellino, R., Havermans, M., Erpelinck, C., Sanders, M. A., Hoogenboezem, R., van de Werken, H. J. G., Rombouts, E., van Lom, K., van Strien, P. M. H., Gebhard, C., et al. (2016). An autonomous CEBPA enhancer specific for myeloid-lineage priming and neutrophilic differentiation. Blood 127, 2991-3003.
31. Bainton, D. F., Ullyot, J. L., and Farquhar, M. G. (1971). The development of neutrophilic polymorphonuclear leukocytes in human bone marrow. J. Exp. Med. 134, 907-934.
32. Bekkering, S. (2013). Another look at the life of a neutrophil. World Journal of Hematology 2, 44.
33. Beyrau, M., Bodkin, J. V., and Nourshargh, S. (2012). Neutrophil heterogeneity in health and disease: a revitalized avenue in inflammation and immunity. Open Biol. 2, 120134.
34. Borregaard, N. (2010). Neutrophils, from marrow to microbes. Immunity 33, 657-670.
35. Chen, H., Lau, M. C., Wong, M. T., Newell, E. W., Poidinger, M., and Chen, J. (2016). Cytofkit: A Bioconductor Package for an Integrated Mass Cytometry Data Analysis Pipeline. PLoS Comput. Biol. 12, e1005112.
36. Doulatov, S., Notta, F., Eppert, K., Nguyen, L. T., Ohashi, P. S., and Dick, J. E. (2010). Revised map of the human progenitor hierarchy shows the origin of macrophages and dendritic cells in early lymphoid development. Nat. Immunol. 11, 585-593.
37. Edvardsson, L., Dykes, J., and Olofsson, T. (2006). Isolation and characterization of human myeloid progenitor populations—TpoR as discriminator between common myeloid and megakaryocyte/erythroid progenitors. Exp. Hematol. 34, 599-609.
38. Elghetany, M. T., Ge, Y., Patel, J., Martinez, J., and Uhrova, H. (2004). Flow cytometric study of neutrophilic granulopoiesis in normal bone marrow using an expanded panel of antibodies: correlation with morphologic assessments. J. Clin. Lab. Anal. 18, 36-41.
39. Friedman, A. D. (2007). Transcriptional control of granulocyte and monocyte development. Oncogene 26, 6816-6828.
40. Kawamura, S., Onai, N., Miya, F., Sato, T., Tsunoda, T., Kurabayashi, K., Yotsumoto, S., Kuroda, S., Takenaka, K., Akashi, K., et al. (2017). Identification of a Human Clonogenic Progenitor with Strict Monocyte Differentiation Potential: A Counterpart of Mouse cMoPs. Immunity 46, 835-848.e4.
41. Kim, M.-H., Yang, D., Kim, M., Kim, S.-Y., Kim, D., and Kang, S. J. (2017). A late-lineage murine neutrophil precursor population exhibits dynamic changes during demand-adapted granulopoiesis. Sci. Rep. 7, 39804.
42. Lee, P. Y., Wang, J.-X., Parisini, E., Dascher, C. C., and Nigrovic, P. A. (2013). Ly6 family proteins in neutrophil biology. J. Leukoc. Biol. 94, 585-594.
43. Levine, J. H., Simonds, E. F., Bendall, S. C., Davis, K. L., Amir, E.-A. D., Tadmor, M. D., Litvin, O., Fienberg, H. G., Jager, A., Zunder, E. R., et al. (2015). Data-Driven Phenotypic Dissection of AML Reveals Progenitor-like Cells that Correlate with Prognosis. Cell 162, 184-197.
44. Lyman, G. H., Abella, E., and Pettengell, R. (2014). Risk factors for febrile neutropenia among patients with cancer receiving chemotherapy: A systematic review. Crit. Rev. Oncol. Hematol. 90, 190-199.
45. Mori, Y., Iwasaki, H., Kohno, K., Yoshimoto, G., Kikushige, Y., Okeda, A., Uike, N., Niiro, H., Takenaka, K., Nagafuji, K., et al. (2009). Identification of the human eosinophil lineage-committed progenitor: revision of phenotypic definition of the human common myeloid progenitor. J. Exp. Med. 206, 183-193.
46. Olsson, A., Venkatasubramanian, M., Chaudhri, V. K., Aronow, B. J., Salomonis, N., Singh, H., and Grimes, H. L. (2016). Single-cell analysis of mixed-lineage states leading to a binary cell fate choice. Nature 537, 698-702.
47. Paul, F., Arkin, Y. 'ara, Giladi, A., Jaitin, D. A., Kenigsberg, E., Keren-Shaul, H., Winter, D., Lara-Astiaso, D., Gury, M., Weiner, A., et al. (2015). Transcriptional Heterogeneity and Lineage Commitment in Myeloid Progenitors. Cell 163, 1663-1677.
48. Pillay, J., den Braber, I., Vrisekoop, N., Kwast, L. M., de Boer, R. J., Borghans, J. A. M., Tesselaar, K., and Koenderman, L. (2010). In vivo labeling with 2H2O reveals a human neutrophil lifespan of 5.4 days. Blood 116, 625-627.
49. Pronk, C. J. H., Rossi, D. J., Mansson, R., Attema, J. L., Norddahl, G. L., Chan, C. K. F., Sigvardsson, M., Weissman, I. L., and Bryder, D. (2007). Elucidation of the phenotypic, functional, and molecular topography of a myeloerythroid progenitor cell hierarchy. Cell Stem Cell 1, 428-442.
50. Radomska, H. S., Huettner, C. S., Zhang, P., Cheng, T., Scadden, D. T., and Tenen, D. G. (1998). CCAAT/enhancer binding protein alpha is a regulatory switch sufficient for induction of granulocytic development from bipotential myeloid progenitors. Mol. Cell. Biol. 18, 4301-4314.
51. R Development Core Team (2016). R: A Language and Environment for Statistical Computing. R Foundation for Statistical Computing, Vienna.
52. Rizzo, M. L. (2016). Statistical Computing with R (CRC Press).
53. Satija, R., Farrell, J. A., Gennert, D., Schier, A. F., and Regev, A. (2015). Spatial reconstruction of single-cell gene expression data. Nat. Biotechnol. 33, 495-502.
54. Silvestre-Roig, C., Hidalgo, A., and Soehnlein, O. (2016). Neutrophil heterogeneity: implications for homeostasis and pathogenesis. Blood 127, 2173-2181.
55. Soehnlein, O., Steffens, S., Hidalgo, A., and Weber, C. (2017). Neutrophils as protagonists and targets in chronic inflammation. Nat. Rev. Immunol. 17, 248-261.
56. Terstappen, L. W., and Loken, M. R. (1990). Myeloid cell differentiation in normal bone marrow and acute myeloid leukemia assessed by multi-dimensional flow cytometry. Anal. Cell. Pathol. 2, 229-240.
57. Zhang, D. E., Zhang, P., Wang, N. D., Hetherington, C. J., Darlington, G. J., and Tenen, D. G. (1997). Absence of granulocyte colony-stimulating factor signaling and neutrophil development in CCAAT enhancer binding protein alpha-deficient mice. Proc. Natl. Acad. Sci. U.S.A 94, 569-574.
58. Zheng, G. X. Y., Terry, J. M., Belgrader, P., Ryvkin, P., Bent, Z. W., Wilson, R., Ziraldo, S. B., Wheeler, T. D., McDermott, G. P., Zhu, J., et al. (2017). Massively parallel digital transcriptional profiling of single cells. Nat. Commun. 8, 14049.

1. A method for treatment of a subject, comprising
(i) processing a biological sample from the subject, the sample being suspected of including neutrophil cells, to determine a concentration level thereof,
(ii) comparing the concentration level to a reference level, and
(iii) treating said subject at least based on said comparison, the treating step including stimulating or inhibiting differentiation of unipotent neutrophil progenitor cells into neutrophil cells so as to modulate the concentration of said neutrophil cells in said subject.

2. A method for evaluating a condition status in a subject, the condition being associated with neutropenia, the method comprising
(i) providing a biological sample from said subject, the sample being suspected of including unipotent neutrophil progenitor cells;
(ii) processing the sample to determine a concentration or activation level of said unipotent neutrophil progenitor cells in said sample;
(iii) comparing the concentration or activation level to a reference level; and
(iv) evaluating the condition status based on at least the comparison in step (iii), the condition being associated with neutropenia.

3. A method for evaluating a cancer in a subject, comprising
(i) providing a biological sample from said subject, the sample being suspected of including unipotent neutrophil progenitor cells;
(ii) processing the sample to determine a concentration or activation level of said unipotent neutrophil progenitor cells in said sample;
(iii) comparing the concentration or activation level to a reference level; and
(iv) evaluating the subject as having or not having cancer based on at least the comparison in step (iii).

4. A method of determining response or resistance to cancer treatment in a subject undergoing cancer treatment, the method comprising
(i) providing a biological sample from said subject, the sample being suspected of including unipotent neutrophil progenitor cells;
(ii) processing the sample to determine a concentration or activation level of said unipotent neutrophil progenitor cells in said sample;
(iii) comparing the concentration or activation level to a reference level; and
(iv) evaluating the response or resistance to the cancer treatment based on at least the comparison in step (iii).

5. A method of determining response or resistance to a treatment for a condition associated with neutropenia in a subject undergoing the treatment, the method comprising
(i) providing a biological sample from said subject, the sample being suspected of including unipotent neutrophil progenitor cells;
(ii) processing the sample to determine a concentration or activation level of said unipotent neutrophil progenitor cells in said sample;
(iii) comparing the concentration or activation level to a reference level; and
(iv) evaluating the response or resistance to the treatment based on at least the comparison in step (iii).

6. A method of reducing risk of cancer progression or cancer relapse in a subject, the method comprising:
(i) providing a biological sample from said subject, the sample being suspected of including unipotent neutrophil progenitor cells;
(ii) processing the sample to determine a concentration or activation level of said unipotent neutrophil progenitor cells in said sample;
(iii) comparing the concentration or activation level to a reference level; and
(iv) selectively administering a cancer therapeutic agent at least based on the comparison in step (iii) so as to reduce risk of cancer progression or cancer relapse in the subject.

7. A method of reducing risk of a condition associated with neutropenia in a subject, the method comprising:
(i) providing a biological sample from said subject, the sample being suspected of including unipotent neutrophil progenitor cells;
(ii) processing the sample to determine a concentration or activation level of said unipotent neutrophil progenitor cells in said sample;
(iii) comparing the concentration or activation level to a reference level, and
(iv) selectively administering a therapeutic agent at least based on the comparison in step (iii) so as to reduce risk of the condition associated with neutropenia in the subject.

8. A method for screening a candidate molecule for an activity on cell differentiation of unipotent neutrophil progenitor cells into neutrophils, the method comprising:
(i) contacting said unipotent neutrophil progenitor cells with the candidate molecule; and
(ii) determining the activity of the candidate molecule on the cell differentiation of said unipotent cells into neutrophils.

9. A method for screening a candidate molecule for an activity on neutrophil differentiation,
(i) providing the candidate molecule
(ii) causing the candidate molecule to contact unipotent neutrophil progenitor cells to determine the activity of the candidate molecule on the cell differentiation of said unipotent cells into neutrophils, and
(iii) receiving information conveying the activity of the candidate molecule on the cell differentiation of said unipotent cells into neutrophils.

10. A method for treatment or prevention of neutropenia in a subject, comprising administering to the subject an effective amount of a purified unipotent neutrophil progenitor cell population.

11. The method of claim 10, wherein said progenitor cells are autologous cells to the subject.

12. A method of inhibiting or preventing tumor growth in a subject, comprising inhibiting differentiation of unipotent neutrophil progenitor cells into neutrophil cells in said subject.

13. A pharmaceutical composition comprising isolated unipotent neutrophil progenitor cells and a pharmaceutically acceptable carrier, wherein said progenitor cells are modified so as to have modified gene expression, modified cell function or to include a ribonucleic acid interference (RNAi) causing molecule, or a conjugated therapeutic agent.

14. The pharmaceutical composition of claim 13, wherein the cells are genetically modified by CRISPR-cas9, lentivirus transduction or RNAi.

15. The method of any one of claims 1 to 7, wherein said biological sample includes blood or a cell fraction thereof.

16. The method of any one of claims 1 to 7, wherein said biological sample includes blood, spleen, tumor tissue or bone marrow, or a cell fraction thereof.

17. The method of any one of claims 1 to 7, wherein said reference level is derived from a cohort of at least 20 reference individuals without disease condition.

18. The method of any one of claims 1 to 7, wherein said reference level is derived from a sample from the subject, the sample being provided prior to or after a treatment performed to treat the subject.

19. The method of claim 1, wherein said subject is afflicted with neutropenia.

20. The method of claim 19, wherein said neutropenia is caused by a cancer.

21. The method of any one of claims 1 to 20, wherein the progenitor cells have at least the phenotype CD45+, CD41−, CD127 (IL-7Rα)−, CD19−, CD3−, CD161 (NK1.1)−, CD169 (Siglec 1)−, CD11c−, Siglec 8−, FcERIα− and CD115 (CSF-1R)−.

22. The method of any one of claims 1 to 20, wherein the progenitor cells have at least the phenotype CD161−, CD34+, CD38+, CD115−, Siglec8−, FcERIα− and CD114+.

23. The method of any one of claims 1 to 20, wherein the progenitor cells have at least the phenotype CD45+, CD235ab−, CD41−, CD127 (IL-7Rα)−, CD19−, CD3−, CD4−, CD161 (NK1.1)−, CD56−, CD169 (Siglec 1)−, CD64−, CD11c−, HLA-DR−, CD86−, CD123−, CD7−, CD10−, CD366−, CD90−, Siglec 8−, FcERIα−, CD115 (CSF-1R)−, CD34+, CD38+, CD45RA+, CD66b+, CD16b+, CD15+, CD114+, CD14int, CD162int, and CD62Lint.

24. The method of any one of claims 1 to 20, wherein the progenitor cells have at least the phenotype hSiglec 8−, hFcεRIα−, hCD3−, hCD7−, hCD10−, hCD11c−, hCD19−, hCD41−, hCD56−, hCD90 (Thy1)−, hCD123 (IL-3Rα)−, hCD125 (IL-5Rα)−, hCD127 (IL-7Rα)−, hCD161−, hCD169−, hCD235a−, hCD66b+, hCD117 (c-Kit)+, hCD38+, and hCD34+.

25. The method of any one of claims 1 to 20, wherein the progenitor cells have at least the phenotype hSiglec 8−, hFcεRIα−, hCD3−, hCD7−, hCD10−, hCD11c−, hCD19−, hCD41−, hCD56−, hCD90 (Thy1)−, hCD123 (IL-3Rα)−, hCD125 (IL-5Rα)−, hCD127 (IL-7Rα)−, hCD161−, hCD169−, hCD235a−, hCD34−, hCD66b+, hCD117 (c-Kit)+, and hCD38+.

26. The method of any one of claims 1 to 25, wherein said subject is human.

27. The method any one of claims 1 to 20, wherein the progenitor cells have at least the phenotype CD161−, CD117(c-Kit)+, Ly6A/E−, CD16/32+, CD115−, SiglecF−, FcERIα− and Ly6G−/lo.

28. The method any one of claims 1 to 20, wherein the progenitor cells have at least the phenotype CD45+, Ter119−, CD41−, CD127 (IL-7Rα)−, CD19- or B220−, CD3−, TCRβ−, CD161 (NK1.1)−, CD335 (NKp46)−, CD169 (Siglec 1)−, F4/80−, CD11c−, MHCII−, CD117 (c-kit)+/int, Ly6A/E (Sca1)−, Siglec F (Siglec 8)−, FcERIα−, CD115 (CSF-1R)−, Ly6C−/int, CD16/32 (FcγRIII/II)+, and Ly6G−/lo.

29. The method any one of claims 1 to 20, wherein the progenitor cells have at least the phenotype CD41−, CD127(IL-7Rα)−, CD3−, CD19−, CD161(NK1.1)−, CD169(Siglec 1)−, CD11c−, Siglec F, FcERIα−, CD115(CSF-1R)−, Ly6A/E(Sca1)−, Ly6G−, CD162(PSGL-1) lo, CD48 lo, Ly6C lo, and CD117(c-Kit)+, CD16/32(FcγRIII/II)+, Ly6B+ and CD11a(LFA1α)+.

30. The method of any one of claims 1 to 20, wherein the progenitor cells have at least the phenotype CD41−, CD127(IL-7Rα)−, CD3, CD19−, CD161(NK1.1)−, CD169(Siglec 1)−, CD11c−, Siglec F, FcERIα−, CD115(CSF-1R)−, Ly6A/E(Sca1)−, CD117(c-Kit)+, CD16/32(FcγRIII/II)+, Ly6B, CD11a(LFA1α)+, and Ly6G+.

31. The method of claims 27 to 30, wherein said subject is a mouse.

32. A kit for cell sorting unipotent neutrophil progenitor cells from a biological sample, the kit comprising detecting agents for CD161, CD34, CD38, CD115, Siglec8, FcERIα and CD114.

33. A kit for cell sorting unipotent neutrophil progenitor cells from a biological sample, the kit comprising detecting agents for CD45, CD41, CD127 (IL-7Rα), CD19, CD3, CD161 (NK1.1), CD169 (Siglec 1), CD11c, Siglec 8, FcERIα and CD115 (CSF-1R).

34. A kit for cell sorting unipotent neutrophil progenitor cells from a biological sample, the kit comprising detecting agents for CD45, CD235ab, CD41, CD127 (IL-7Rα), CD19, CD3, CD4, CD161 (NK1.1), CD56, CD169 (Siglec 1), CD64, CD11c, HLA-DR, CD86, CD123, CD7, CD10, CD366, CD90, Siglec 8, FcERIα, CD115 (CSF-1R), CD34, CD38, CD45RA, CD66b, CD16b, CD15, CD114, CD14, CD162, and CD62L.

35. A kit for cell sorting unipotent neutrophil progenitor cells from a biological sample, the kit comprising detecting agents for hSiglec 8, hFcεRIα, hCD3, hCD7, hCD10, hCD11c, hCD19, hCD41, hCD56, hCD90 (Thy1), hCD123 (IL-3Rα), hCD125 (IL-5Rα), hCD127 (IL-7Rα), hCD161, hCD169, hCD235a, hCD66b, hCD117 (c-Kit), hCD38, and hCD34.

36. A kit for cell sorting unipotent neutrophil progenitor cells from a biological sample, the kit comprising detecting agents for CD41, CD127(IL-7Rα), CD3, CD19, CD161(NK1.1), CD169(Siglec 1), CD11c, Siglec F, FcERIα, CD115(CSF-1R), Ly6A/E(Sca1), Ly6G, CD162(PSGL-1), CD48, Ly6C, and CD117(c-Kit), CD16/32(FcγRIII/II), Ly6B and CD11a(LFA1α).

37. A kit for cell sorting unipotent neutrophil progenitor cells from a biological sample, the kit comprising detecting agents for CD41, CD127(IL-7Rα), CD3, CD19, CD161(NK1.1), CD169(Siglec 1), CD11c, Siglec F, FcERIα, CD115(CSF-1R), Ly6A/E(Sca1), CD117(c-Kit), CD16/32(FcγRIII/II), Ly6B, CD11a(LFA1α), and Ly6G.

38. The kit of any one of claims 32 to 37, wherein said biological sample includes blood or a cell fraction thereof.

39. The kit of any one of claims 32 to 37, wherein said biological sample includes blood, spleen, tumor tissue or bone marrow, or a cell fraction thereof.
